(19) United States
(12) Patent Application Publication
(10) Pub. No.: US 2017/0022280 A1
(43) Pub. Date: Jan. 26, 2017

(54) METHOD FOR TREATING ANTIBODY-MEDIATED REJECTION POST-TRANSPLANTATION

(71) Applicant: Cedars-Sinai Medical Center, Los Angeles, CA (US)

(72) Inventors: Stanley C. Jordan, Manhattan Beach, CA (US); Ashley Vo, Northridge, CA (US); Jua Choi, Porter Ranch, CA (US)

(73) Assignee: Cedars-Sinai Medical Center, Los Angeles, CA (US)

(21) Appl. No.: 15/219,121

(22) Filed: Jul. 25, 2016

Related U.S. Application Data
(60) Provisional application No. 62/196,806, filed on Jul. 24, 2015.

(51) Int. Cl. C07K 16/28

(52) U.S. Cl. CPC C07K 16/2866; C07K 16/2887; C07K 2317/24; C07K 2317/76; A61K 2039/505

(57) ABSTRACT

Described herein are methods for treating antibody mediated rejection of transplanted organs using inhibitors of IL-6. In one embodiment, the IL-6 inhibitor is Tocilizumab and is administered simultaneously or sequentially with intravenous immunoglobulin (IVIG).

### CROSS-REFERENCE TO RELATED APPLICATION

This application claims the benefit under 35 U.S.C. §119(e) of U.S. Provisional Patent Application No. 62/196,806 filed on Jul. 25, 2015, which is incorporated herein by reference in its entirety.

### TECHNICAL FIELD

The invention relates to treatments for antibody-mediated transplant rejection.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Antibody-mediated rejection (ABMR) is a unique, significant and often severe form of allograft rejection that is not amenable to treatment with standard-of-care immunosuppressive medications. Significant advances have occurred in our ability to predict patients at risk for and to diagnose ABMR. These include the development of newer anti-HLA antibody detection techniques, especially those detecting C1q (complement) binding donor-specific antibodies (DSAs) and assays for non-HLA antibodies associated with ABMR. The pathophysiology of ABMR suggests a prime role for antibodies, B-cells and plasma cells, but other effector molecules, especially the complement system, point to potential targets that could modify the ABMR process.

ABMR continues to be observed in 30-40% cases, comprising the primary cause of early graft loss. In addition, transplant glomerulopathy (TG) is a known consequence of persistent DSA positivity which rapidly dissipates allograft function resulting in graft failure and return to dialysis with attendant emotional consequences for the patients and financial consequences for the health care system.

Therapies for complement dependent (C4d+) ABMR are limited. Currently accepted therapies include high dose IVIG+/−rituximab, plasma exchange (PLEX) with low dose IVIG+/−rituximab and a minimal experience with bortezomib. No current therapy is FDA approved and patients are often treated with combination therapies that make analysis of efficacy of any one agent difficult.

Based on these observations, there is a large unmet clinical need for new therapies to treat ABMR. This is also true for CABMR and Transplant Glomerulopathy (TG) that are now leading causes of chronic allograft failure.

### SUMMARY

The following embodiments and aspects thereof are described and illustrated in conjunction with systems, compositions and methods which are meant to be exemplary and illustrative, not limiting in scope.

Provided herein are methods for treating, inhibiting and/or reducing the severity of antibody mediated rejection (ABMR) of an organ transplant in a subject in need thereof. The methods include providing an inhibitor of IL-6 and administering an effective amount of the IL-6 inhibitor to the subject in need thereof, so as to treat, inhibit, and/or reduce the severity of ABMR in the subject. In one embodiment, the subject has undergone standard-of-care treatment for ABMR and the subject's response to standard-of-care treatment is ineffective.

Also provided herein are methods for treating, inhibiting and/or reducing the severity of ABMR of an organ transplant in a subject in need thereof. The methods include providing an inhibitor of IL-6 and administering an effective amount of the IL-6 inhibitor to the subject in need thereof, so as to treat, inhibit and/or reduce the severity of ABMR of an organ transplant in the subject.

Further provided herein are methods for reducing and/or eliminating donor specific HLA antibodies in a subject that has undergone organ transplant. The methods include providing an inhibitor of IL-6 and administering an effective amount of the IL-6 inhibitor to the subject, so as to reduce and/or eliminate donor specific HLA antibodies in the subject

Also provided herein are methods for treating, inhibiting and/or reducing the severity of ABMR post-organ transplant in highly HLA-sensitized patients. The methods include providing an inhibitor of IL-6 and administering an effective amount of the IL-6 inhibitor to the subject, so as to treat, inhibit and/or reduce the severity of ABMR post-organ transplant in highly HLA-sensitized patients.

In some embodiments, the subject has undergone an organ transplant and exhibits symptoms of antibody mediated rejection (ABMR) of the transplanted organ.

In one embodiment, the IL-6 inhibitor is administered during transplant. In another embodiment, the IL-6 inhibitor is administered after transplant. In further embodiments, the IL-6 inhibitor is administered sequentially or simultaneously with the standard-of-care treatment.

In exemplary embodiments, the organ is one or more of heart, liver, lungs, pancreas or intestines. In one embodiment, the organ is the kidneys.

In exemplary embodiments, if the organ transplanted is kidney, the symptoms of ABMR are any one or more of: (i) deterioration of allograft function measured by serum Creatinin and estimated Glomerular filtration rate (eGFR); (ii) presence of donor-specific antibodies; (iii) biopsy evidence of capillaritis, inflammation and complement (C4d) deposition, or (iv) combinations thereof.

In exemplary embodiments, the IL-6 inhibitor is selected from the group consisting of a small molecule, a peptide, an antibody or a fragment thereof and a nucleic acid molecule. In one embodiment, the IL-6 inhibitor inhibits the receptor of IL-6 (IL-6R). In an embodiment, the inhibitor is Tocilizumab. In some embodiments, Tocilizumab is administered simultaneously or sequentially with intravenous immunoglobulin (IVIG).

In one embodiment, the standard-of-care treatment comprises intravenous immunoglobulin and rituximab.

In some embodiments, the IL-6 inhibitor is administered intravenously or subcutaneously. In exemplary embodiments, if the IL-6 inhibitor (for example, Tocilizumab) is administered intravenously, the effective amount of the IL-6 inhibitor is at a dose of about 4-8 mg/kg/month, about 3-8 mg/kg/month, about 1-4 mg/kg/month, about 1-5 mg/kg/month, about 5-10 mg/kg/month or combinations thereof. In exemplary embodiments, if the IL-6 inhibitor (for example, Tocilizumab) is administered subcutaneously, the effective amount of the IL-6 inhibitor is at a dose of about 150-170 mg every two weeks if the subject weighs 100 kg or less and about 150-170 mg per week if the subject weights more than 100 kg. In one embodiment, if the IL-6 inhibitor (for example, Tocilizumab) is administered subcutaneously, the effective amount of the IL-6 inhibitor is about 162 mg every two weeks if the subject weighs 100 kg or less and about 162 mg per week if the subject weights more than 100 kg.

In various embodiments, the IL-6 inhibitor is administered for any one or more of about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 24 months, about 30 months, about 36 months or combinations thereof.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1A-FIG. 1B depict in accordance with various embodiments of the invention, (FIG. 1A) IL-6 expression in kidney tissue showing that IL-6 is extensively expressed in ABMR. (FIG. 1B) IL-6+ cells are significantly increased in allografts with ABMR. Morphometric analysis of IL-6 staining in native kidneys (native, n=6 with thin basement membrane disease), transplants without rejection (tx, n=9), transplants with cell mediated rejection (CMR, n=12) and antibody-mediated rejection (ABMR, n=11).

FIG. 2 depicts in accordance with various embodiments of the invention, cytokine levels pre-, at- and post-biopsy (Bx) in HLA-sensitized patients who had graft injuries and those post-transplant in stable HS patients. AMR: antibody-mediated rejection; CMR: cell-mediated rejection; ATN: acute tubular necrosis; CNI: calcineurin inhibitor.

FIG. 3A-FIG. 3B depict in accordance with various embodiments of the invention, (FIG. 3A) anti-IL6R suppresses plasma cells Ig production in HLA allosensitized mouse model. (FIG. 3B) anti-IL6R inhibits anti-HLA A2 IgG production in bone marrow cells from sensitized animals.

FIG. 4A-FIG. 4B depict in accordance with various embodiments of the invention, (FIG. 4A) status of highly sensitized patients with severe ABMR post anti-IL-6R treatment. *Relative Intensity Scale (RIS) [0 points=No DSA; 2 points=<5000 MFI {weak}; 5 points=5000-10,000 MFI {moderate}; 10 points=>10,000 MFI {strong}]. (FIG. 4B) average donor specific antibody (DSA) RIS score decreases from baseline to 12 months post initiation of Tocilizumab. The average DSA RIS at 12M has decreased significantly compared to baseline, and this is statistically significant with a p-value of 0.065.

FIG. 5A-FIG. 5B depict in accordance with various embodiments of the invention, (FIG. 5A) immunodominant donor specific antibody (DSA) scores for ABMR patients treated with TCZ (n=18); Relative Intensity Scale (RIS) [0 points=No DSA; 2 points=<5000 MFI {weak}; 5 points=5000-10,000 MFI {moderate}; 10 points=>10,000 MFI {strong}]. (FIG. 5B) Mean immunodominant DSA values for TCZ treated patients.

FIG. 6A-FIG. 6B depict in accordance with various embodiments of the invention, (FIG. 6A) kidney transplant survival in patients with no ABMR v. severe ABMR and standard-of-care treatment v. Severe ABMR+ TCZ. This graph shows the long-term graft survival among patients with severe ABMR treated with standard-of-care (IVIG+Rituximab+/−PLEX) treatment, no ABMR, and severe ABMR treated with TCZ. TCZ significantly improves graft survival in our patients with ABMR over 4 years of treatment. (FIG. 6B) Kidney transplant survival in patients with transplant glomerulopathy (TG): TCZ v. standard-of-care treatment only. This graph shows the long-term graft survival among patients treated with Transplant Glomerulopathy (TG). This is a disease mediated by DSAs and has a poor outcome. As can be seen, TCZ significantly improves graft survival in our patients with TG over 4 years of treatment.

FIG. 7 depicts in accordance with various embodiments of the invention, average serum Creatinine (mg/dl) to assess kidney function, post Tocilizumab treatment. Serum creatinine (mg/dl) remains stable from initiation of Tocilizumab to up to 36M post Tocilizumab treatments (p-value=NS).

FIG. 8A-FIG. 8C depict in accordance with various embodiments of the invention, Kaplan-Meier curves of kidney allograft survival by treatment after ABMR (Tocilizumab (TCZ) vs Standard of care (SOC)) (FIG. 8A), patient survival by treatment after ABMR (FIG. 8B) and kidney allograft survival by treatment after TG biopsy (FIG. 8C). FIG. 8A shows the classic approach to determine kidney allograft survival based on treatment (Tocilizumab vs SOC) after ABMR biopsy. FIG. 8B shows patient survival based on treatment (Tocilizumab vs SOC) after ABMR biopsy. The FIG. 8C shows the kidney allograft survival based on treatment after TG biopsy. Abbreviations: TCZ, Tocilizumab; SOC, Standard of care.

FIG. 9A-FIG. 9F depict in accordance with various embodiments of the invention, the kidney allograft biopsy phenotypes before and after treatment by Tocilizumab. Abbreviations: TCZ, Tocilizumab; TG, Transplant Glomerulopathy; IFTA, Interstitial fibrosis and tubular atrophy. * Biopsy analysis performed in 9 patients with available and adequate biopsy before and after treatment.

FIG. 10 depicts in accordance with various embodiments of the invention, variables associated with allograft survival. Random forest using Minimal Depth variable selection. This figure shows the random survival forest using minimal Depth variable selection. Low minimal depth indicates important variables. The dashed line is the threshold of maximum value for variable selection. Abbreviations: TCZ, Tocilizumab; ECD, Expanded criteria donor; CIT, Cold ischemia time; DGF, Delayed graft function; DSA, Donor Specific antibodies, eGFR, estimated Glomerular Filtration.

FIG. 11 depicts in accordance with various embodiments of the invention, variables associated with allograft survival. Random forest Variable Importance (VIMP) selection. This shows the random forest Variable Importance (VIMP). Black (blue) bars indicates positive VIMP, grey (red) indicates negative VIMP. Importance is relative to positive length of bars. Abbreviations: TCZ, Tocilizumab; ECD, Expanded criteria donor; CIT, Cold ischemia time; DGF, Delayed graft function; DSA, Donor Specific antibodies, eGFR, estimated Glomerular Filtration.

FIG. 12 depicts in accordance with various embodiments of the invention, comparison of Minimal Depth and Vimp rankings. This shows the comparison between Minimal Depth and Vimp rankings. Points on the red dashed line are ranked equivalently, points above have higher VIMP ranking, those below have higher minimal depth ranking. Abbreviations: TCZ, Tocilizumab; ECD, Expanded criteria donor; CIT, Cold ischemia time; DGF, Delayed graft function; DSA, Donor Specific antibodies, eGFR, estimated Glomerular Filtration.

FIG. 13 depicts in accordance with various embodiments of the invention, Kaplan-Meier curves of kidney allograft survival by treatment after ABMR (Tocilizumab (TCZ) vs Standard of care (SOC)) when graft loss was censored for those who retained their grafts for 3 months post biopsy. Abbreviations: TCZ, Tocilizumab; SOC, Standard of care.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

The term “sample” or “biological sample” as used herein denotes a sample taken or isolated from a biological organism, e.g., a fluid sample from a subject. Exemplary biological samples include, but are not limited to, cheek swab; mucus; whole blood, blood, serum; plasma; urine; saliva; semen; lymph; fecal extract; sputum; other body fluid or biofluid; cell sample; tissue sample; tumor sample; and/or tumor biopsy etc. The term also includes a mixture of the above-mentioned samples. The term “sample” also includes untreated or pretreated (or pre-processed) biological samples. In some embodiments, a sample can comprise one or more cells from the subject. In some embodiments, a sample can be a tumor cell sample, e.g. the sample can comprise cancerous cells, cells from a tumor, and/or a tumor biopsy.

The term “statistically significant” or “significantly” refers to statistical evidence that there is a difference. It is defined as the probability of making a decision to reject the null hypothesis when the null hypothesis is actually true. The decision is often made using the p-value.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with, a disease or disorder. The term “treating” includes reducing or alleviating at least one adverse effect or symptom of a condition, disease or disorder, such as ABMR. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease also includes providing relief from the symptoms or side-effects of the disease (including palliative treatment).

“Beneficial results” or “desired results” may include, but are in no way limited to, lessening or alleviating the severity of the disease condition, preventing the disease condition from worsening, curing the disease condition, preventing the disease condition from developing, lowering the chances of a patient developing the disease condition, decreasing morbidity and mortality, and prolonging a patient's life or life expectancy. As non-limiting examples, “beneficial results” or “desired results” may be alleviation of one or more symptom(s), diminishment of extent of the deficit, stabilized (i.e., not worsening) state of cancer progression, delay or slowing of metastasis or invasiveness, and amelioration or palliation of symptoms associated with the cancer.

As used herein, the term “administering,” refers to the placement an agent as disclosed herein into a subject by a method or route which results in at least partial localization of the agents at a desired site

As used herein, the term “antibody” refers to an intact immunoglobulin or to a monoclonal or polyclonal antigen-binding fragment with the Fc (crystallizable fragment) region or FcRn binding fragment of the Fc region, referred to herein as the “Fc fragment” or “Fc domain”. Antigen-binding fragments may be produced by recombinant DNA techniques or by enzymatic or chemical cleavage of intact antibodies. Antigen-binding fragments include, inter alia, Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The Fc domain includes portions of two heavy chains contributing to two or three classes of the antibody. The Fc domain may be produced by recombinant DNA techniques or by enzymatic (e.g. papain cleavage) or via chemical cleavage of intact antibodies.

The term “antibody fragment,” as used herein, refer to a protein fragment that comprises only a portion of an intact antibody, generally including an antigen binding site of the intact antibody and thus retaining the ability to bind antigen. Examples of antibody fragments encompassed by the present definition include: (i) the Fab fragment, having VL, CL, VH and CH1 domains; (ii) the Fab′ fragment, which is a Fab fragment having one or more cysteine residues at the C-terminus of the CH1 domain; (iii) the Fd fragment having VH and CH1 domains; (iv) the Fd′ fragment having VH and CH1 domains and one or more cysteine residues at the C-terminus of the CH1 domain; (v) the Fv fragment having the VL and VH domains of a single arm of an antibody; (vi) the dAb fragment (Ward et al., Nature 341, 544-546 (1989)) which consists of a VH domain; (vii) isolated CDR regions; (viii) F(ab′)2 fragments, a bivalent fragment including two Fab′ fragments linked by a disulphide bridge at the hinge region; (ix) single chain antibody molecules (e.g., single chain Fv; scFv) (Bird et al., Science 242:423-426 (1988); and Huston et al., PNAS (USA) 85:5879-5883 (1988)); (x) “diabodies” with two antigen binding sites, comprising a heavy chain variable domain (VH) connected to a light chain variable domain (VL) in the same polypeptide chain (see, e.g., EP 404,097; WO 93/11161; and Hollinger et al., Proc. Natl. Acad. Sci. USA, 90:6444-6448 (1993)); (xi) “linear antibodies” comprising a pair of tandem Fd segments (VH-CH1-VH-CH1) which, together with complementary light chain polypeptides, form a pair of antigen binding regions (Zapata et al. Protein Eng. 8(10):1057-1062 (1995); and U.S. Pat. No. 5,641,870).

As used herein, “selectively binds” or “specifically binds” refers to the ability of an antibody or antibody fragment thereof described herein to bind to a target, such as a molecule present on the cell-surface, with a KD 10⁻⁵ M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. Specific binding can be influenced by, for example, the affinity and avidity of the polypeptide agent and the concentration of polypeptide agent. The person of ordinary skill in the art can determine appropriate conditions under which the polypeptide agents described herein selectively bind the targets using any suitable methods, such as titration of a polypeptide agent in a suitable cell binding assay.

As used herein, “ineffective” treatment refers to when a subject is administered a treatment and there is less than 1%, 5%, 10%, 15%, 20%, 25%, 30%, 40%, 50%, 60%, 70%, 80%, 90% or 95% improvement in symptoms. In exemplary embodiments, standard-of-care treatment for kidney transplant is ineffective if there is less than 1%, 5%, 10%, 15%, 20%, 25%, 30%, 40%, 50%, 60%, 70%, 80%, 90% or 95% improvement in ABMR.

Interleukin-6 is an important mediator of inflammation and the development, maturation, and activation of T-cells, B-cells and plasma cells. Excessive IL-6 production has been linked to a number of human diseases characterized by excessive and unregulated antibody production and autoimmunity. Tocilizumab (Actemra®, Roche/Genentech, CA, USA) is the first in class humanized monoclonal aimed at the IL-6 receptor (IL-6R). Tocilizumab binds to both soluble and membrane bound forms of the IL-6R receptor. Tocilizumab was recently approved by the FDA for treatment of rheumatoid arthritis and juvenile idiopathic arthritis.

The inventors found that Tocilizumab improved transplant rates in patients who had failed desensitization with standard-of-care treatment which includes intravenous immunoglobulin (IVIG) and rituximab with or without plasma exchange (PLEX). Donor specific antibodies (DSAs) that were resistant to other therapies were significantly reduced with therapy and none of the transplanted patients showed antibody mediated rejection post-transplant. Accordingly, provided herein are methods for treating antibody-mediated rejection post-transplantation in subjects that have undergone an organ transplant.

Provided herein is a method for treating, inhibiting and/or reducing the severity of antibody mediated rejection (ABMR) of an organ transplant in a subject in need thereof. The method comprises, consists or consists essentially of administering standard-of-care treatment to the subject and assessing the subject for ABMR. In some embodiments, if there is no improvement in ABMR after administering the standard-of-care treatment, the method further comprises providing an inhibitor of IL-6 and administering an effective amount of the IL-6 inhibitor so as to treat ABMR in a subject. In one embodiment, the IL-6 inhibitor is administered after organ transplant. In one embodiment, the IL-6 inhibitor is Tocilizumab, which is administered at dosages and frequencies described herein. In one embodiment, the organ transplant is a kidney transplant.

Also provided herein is a method for treating, inhibiting and/or reducing the severity of ABMR of an organ transplant in a subject in need thereof. The method comprises, consists or consists essentially of administering standard-of-care treatment to the subject; providing an inhibitor of IL-6 and administering an effective amount of the inhibitor so as to treat ABMR in a subject. In some embodiments, the standard of care treatment and the IL-6 inhibitor are administered sequentially or simultaneously. In one embodiment, the IL-6 inhibitor is administered after organ transplant. In one embodiment, the IL-6 inhibitor is Tocilizumab, which is administered at dosages and frequencies described herein. In one embodiment, the organ transplant is a kidney transplant.

Further provided herein is a method for treating ABMR of an organ transplant in a subject in need thereof. The method comprises, consists or consists essentially of providing an inhibitor of IL-6 and administering an effective amount of the inhibitor to the subject that has undergone organ transplant. In some embodiments, the methods include administering standard-of-care treatment to the subject before administering the IL-6 inhibitor, wherein the IL-6 inhibitor is administered to the subject if the subject does not respond to standard-of-care treatment. In one embodiment, the inhibitor is administered during (concurrently with) organ transplantation. In another embodiment, the IL-6 inhibitor is administered after organ transplantation. In a further embodiment, the inhibitor is administered during and after organ transplantation. In an embodiment, the subject has not been administered the IL-6 inhibitor prior to organ transplant. In one embodiment, the IL-6 inhibitor is Tocilizumab, which is administered at dosages and frequencies described herein. In one embodiment, the organ transplant is a kidney transplant.

Also provided herein is a method for treating, inhibiting and/or reducing the severity of ABMR of an organ transplant in a subject that has undergone organ transplant and does not respond to standard of care treatment. The method comprises, consists or consists essentially of providing an inhibitor of IL-6 and administering an effective amount of the inhibitor to the subject that has undergone organ transplant and does not respond to standard of care treatment. In one embodiment, the IL-6 inhibitor is Tocilizumab, which is administered at dosages and frequencies described herein. In one embodiment, the organ transplant is a kidney transplant.

Further provided herein is a method for treating, inhibiting and/or reducing the severity of ABMR of an organ transplant in a subject in need thereof. The method comprises, consists or consists essentially of providing an inhibitor of IL-6 and administering an effective amount of the inhibitor to the subject that has undergone organ transplant. In one embodiment, the inhibitor is administered during (concurrently with) organ transplantation. In another embodiment, the inhibitor is administered after organ transplantation. In a further embodiment, the inhibitor is administered during and after organ transplantation. In an embodiment, the subject has not been administered the IL-6 inhibitor prior to organ transplant. In one embodiment, the IL-6 inhibitor is Tocilizumab, which is administered at dosages and frequencies described herein. In one embodiment, the organ transplant is a kidney transplant.

Further provided herein is a method for treating, inhibiting and/or reducing the severity of ABMR of kidney transplant in a subject in need thereof. The method comprises, consists or consists essentially of providing an inhibitor of IL-6 and administering an effective amount of the inhibitor to the subject that has undergone kidney transplant. In some embodiments, the methods include administering standard-of-care treatment to the subject before administering the IL-6 inhibitor, wherein the IL-6 inhibitor is administered to the subject if the subject does not respond to standard-of-care treatment. In one embodiment, the inhibitor is administered during organ transplantation. In another embodiment, the inhibitor is administered after organ transplantation. In a further embodiment, the inhibitor is administered during and after organ transplantation. In an embodiment, the subject has not been administered the IL-6 inhibitor prior to organ transplant. In one embodiment, the IL-6 inhibitor is Tocilizumab, which is administered at dosages and frequencies described herein. In exemplary embodiments, in subjects with kidney transplant, AMBR is defined as (i) deterioration of allograft function in a high-risk transplant recipient (i.e. sensitized patient with history of DSAs) measured by serum Cr (creatinine) and estimated Glomerular filtration rate (eGFR) (defined as a decline >20% from baseline); (ii) association with the presence of DSA (usually increasing in strength) measured by luminex techniques; and/or (iii) biopsy evidence of capillaritis, inflammation and CD4 deposition.

Further provided herein is a method for reducing and/or eliminating donor specific HLA antibodies in a subject that has undergone organ transplant. The method comprises, consists of or consists essentially of providing an inhibitor of IL-6 and administering an effective amount of the composition to the subject so as to reduce and/or eliminate donor specific HLA antibodies. In one embodiment, reducing and/or eliminating donor specific HLA antibodies treats, inhibits and/or reduces the severity of ABMR in the subject. In one embodiment, the IL-6 inhibitor is Tocilizumab, which is administered at dosages and frequencies described herein. In one embodiment, the organ transplant is a kidney transplant.

Also provided herein is a method for treating, inhibiting and/or reducing the severity of ABMR post-organ transplant in highly-HLA sensitized patients. The method comprises, consists of or consists essentially of providing an inhibitor of IL-6 and administering an effective amount of the composition to the subject so as to treat, inhibit and/or reduce the severity of ABMR post-organ transplant in highly-HLA sensitized patients. In one embodiment, the IL-6 inhibitor is Tocilizumab, which is administered at dosages and frequencies described herein. In one embodiment, the organ transplant is a kidney transplant.

In various embodiments of the methods described herein, the standard-of-care treatment for ABMR comprises administering an effective amount of intravenous immunoglobulin (IVIG) and rituximab, as will be apparent to a person of skill in the art.

In various embodiments of the methods described herein, the IL-6 inhibitor directly or indirectly inhibits IL-6. In some embodiments of the methods described herein, the IL-6 inhibitor directly inhibits IL-6 and is selected from the group consisting of a small molecule, a peptide, an antibody or a fragment thereof that specifically binds IL-6 or IL-6R and a nucleic acid molecule. In one embodiment of the methods described herein, the IL-6 inhibitor indirectly inhibits IL-6 via IL-6 receptor (IL-6R) wherein inhibitor of IL-6R is selected from the group consisting of a small molecule, a peptide, an antibody or a fragment thereof and a nucleic acid molecule. In some embodiments of the methods described herein, the nucleic acid molecule is a siRNA molecule specific for IL-6 or IL-6R. In some embodiments, the inhibitor is a bispecific molecule that specifically binds IL-6 and IL6R, so as to inhibit IL-6. In some embodiments of the methods described herein, the antibody is selected from the group consisting of monoclonal antibody or fragment thereof, a polyclonal antibody or a fragment thereof, chimeric antibodies, humanized antibodies, human antibodies, and a single chain antibody. In an embodiment of the methods described herein, the inhibitor is Tocilizumab, which is an anti-IL-6R antibody. In some embodiments, Tocilizumab is sequentially or simultaneously administered with intravenous immunoglobulin (IVIG) and/or rituximab.

Further provided herein are methods for detecting ABMR in a subject that has undergone an organ transplant. The methods include providing a sample from the subject, detecting the levels of a diagnostic marker indicative of ABMR, wherein an increase in the levels of the diagnostic marker relative to a reference value is indicative ABMR. In some embodiments, if a subject is diagnosed with ABMR, the method includes administering an effective amount of an inhibitor of IL-6 so as to treat, inhibit, and/or reduce the severity of ABMR. In some embodiments, the IL-6 inhibitor is Tocilizumab. In one embodiment, the organ transplant is a kidney transplant and the diagnostic markers are any one or more of serum creatinine levels, estimated Glomerular filtration rate (eGFR) or combinations thereof. In some embodiments, the organs are any of pancreas, heart, lung, and liver and the ABMR diagnostic marker is C4D deposition in affected areas. In some embodiments, the reference value refers to the mean or median level of the diagnostic markers in a subject that has not undergone organ transplant. In some embodiments, the reference value refers to the mean or median level of the diagnostic markers in a subject that has undergone organ transplant but does not have ABMR of the transplanted organ. In some embodiments, the reference value refers to the mean or median level of the diagnostic markers in a subject that has undergone organ transplant, has been treated for ABMR of the transplanted organ and has overcome ABMR of the transplanted organ. In one embodiment, the marker includes donor specific HLA antibodies. In some embodiments, in subjects that have undergone kidney transplant, the levels of the HLA antibodies, serum creatinine levels and/or eGFR is increased by at least about 10%, 20%, 30%, 40%, 50%, 60%, 70%, 80% 90% or 100% or combinations thereof. In some embodiments, in subjects that have undergone kidney transplant, the levels of the HLA antibodies, serum creatinine levels and/or eGFR is increased by at least about 1-fold, 2-fold, 3-fold, 4-fold, 5-fold, 6-fold, 7-fold, 8-fold, 9-fold, 10-fold, 15-fold, 20-fold, 30-fold, 50-fold, 15-fold, 100-fold or combinations thereof. In various embodiments, the sample is obtained

In exemplary embodiments, the organs are any one or more of heart, liver, kidneys, lungs, pancreas or intestines. In an embodiment, the organ is kidneys.

### Dosages of the Invention

In some embodiments of the invention, the effective amounts of IL-6 inhibitor in the composition can be in the range of about 10-50 mg/day, 50-100 mg/day, 100-150 mg/day, 150-200 mg/day, 100-200 mg/day, 200-300 mg/day, 300-400 mg/day, 400-500 mg/day, 500-600 mg/day, 600-700 mg/day, 700-800 mg/day, 800-900 mg/day, 900-1000 mg/day, 1000-1100 mg/day, 1100-1200 mg/day, 1200-1300 mg/day, 1300-1400 mg/day, 1400-1500 mg/day, 1500-1600 mg/day, 1600-1700 mg/day, 1700-1800 mg/day, 1800-1900 mg/day, 1900-2000 mg/day, 2000-2100 mg/day, 2100-2200 mg/day, 2200-2300 mg/day, 2300-2400 mg/day, 2400-2500 mg/day, 2500-2600 mg/day, 2600-2700 mg/day, 2700-2800 mg/day, 2800-2900 mg/day or 2900-3000 mg/day. In one embodiment of the invention, the IL-6 inhibitor is Tocilizumab, which binds IL-6R.

In further embodiments of the invention, the effective amount of IL-6 inhibitor for use with the claimed methods may be in the range of 1-5 mg/kg, 5-10 mg/kg, 10-50 mg/kg, 50-100 mg/kg, 100-150 mg/kg, 150-200 mg/kg, 100-200 mg/kg, 200-300 mg/kg, 300-400 mg/kg, 400-500 mg/kg, 500-600 mg/kg, 600-700 mg/kg, 700-800 mg/kg, 800-900 mg/kg or 900-1000 mg/kg. In one embodiment of the invention, the IL-6 inhibitor is Tocilizumab, which binds IL-6R.

In additional embodiments, the effective amount of IL-6 inhibitor is about 1-2 mg/kg, 2-3 mg/kg, 3-4 mg/kg, 4-5 mg/kg, 5-6 mg/kg, 6-7 mg/kg, 7-8 mg/kg, 8-9 mg/kg, 9-10 mg/kg, 10-11 mg/kg, 11-12 mg/kg, 12-13 mg/kg, 13-15 mg, 15-20 mg/kg or 20-25 mg/kg. In one embodiment, the IL-6 inhibitor is Tocilizumab, which binds IL-6R. In one embodiment, these dosages are administered when the mode of administration is intravenous.

In additional embodiments, the effective amount of the IL-6 inhibitor is any one or more of about 100-125 mg, 125-150 mg, 150-175 mg, 160-170 mg, 175-200 mg, 155-165 mg, 160-165 mg, 165-170 mg, 155-170 mg, or combinations thereof, every two weeks if the subject weights less than 100 kg. In one embodiment, the effective amount is 162 mg every two weeks if the subject weights less than 100 kg.

In additional embodiments, the effective amount of the IL-6 inhibitor is any one or more of about 100-125 mg, 125-150 mg, 150-175 mg, 160-170 mg, 175-200 mg, 155-165 mg, 160-165 mg, 165-170 mg, 155-170 mg, or combinations thereof every week if the subject weights more than 100 kg. In one embodiment, the effective amount is 162 mg every week if the subject weights more than 100 kg.

In various embodiments, the IL-6 inhibitor (for example Tocilizumab) is administered at any one or more of the dosages described herein at least once 1-7 times per week, 1-7 times per month, 5-10 times per month or combinations thereof for 1 month, 2 months, 3 months, 4 months, 5 months 6 months, 7 months, 8 months, 9 months, 10 months, 11 months, 12 months, 14 months, 16 months, 18 months, 20 months, 22 months, 24 months or combinations thereof. In some embodiments, Tocilizumab is administered at 4-8 mg/kg per month for 3-9 months. In some embodiments, Tocilizumab is administered at 5-8 mg/kg per month for 3-9 months. In some embodiments, Tocilizumab is administered at 6-8 mg/kg per month for 3-9 months. In some embodiments, Tocilizumab is administered at 7-8 mg/kg per month for 3-9 months. In some embodiments, Tocilizumab is administered at 4-8 mg/kg per month for 3-11 months.

Typical dosages of an effective amount of a IL-6 inhibitor, such as Tocilizumab, can be in the ranges recommended by the manufacturer where known therapeutic compounds are used, and also as indicated to the skilled artisan by the in vitro responses or responses in animal models. For example, Tocilizumab is currently recommended at 8 mg/kg (max dose 800 mg) IV q month×12M starting at day 2 post diagnosis of ABMR. The same or similar dosing can be used in accordance with various embodiments of the present invention, or an alternate dosage may be used in connection with alternate embodiments of the invention. The actual dosage can depend upon the judgment of the physician, the condition of the patient, and the effectiveness of the therapeutic method based, for example, on the in vitro responsiveness of relevant cultured cells or histocultured tissue sample, or the responses observed in the appropriate animal models

In some embodiments, effective amount of Tocilizumab is 8 mg/kg (max dose 800 mg) IV q month×12M starting at day 2 post diagnosis of ABMR. Patient may be pre-medicated 30 minutes before with acetaminophen oral, diphenhydramine oral, and solumedrol 40 mg IV push. Tocilizumab may be diluted to 100 mL by a healthcare professional with sterile 0.9% w/v sodium chloride solution using aseptic technique Tocilizumab is recommended for IV infusion over 1 hour. For individuals whose body weight is more than 100 kg, doses exceeding 800 mg per infusion are not recommended. Dose may be rounded to the nearest 80 mg, 200 mg, and/or 400 mg vial.

In various embodiments, the IL-6 inhibitor (for example, Tocilizumab) is administered during organ transplants and up to any one or more of one month, two months, six months, twelve months, 18 months, 24 months or 30 months after transplant.

### Pharmaceutical Composition

In various embodiments, the present invention provides a pharmaceutical composition. The pharmaceutical composition includes an inhibitor of IL-6. In an embodiment, the IL-6 inhibitor directly or indirectly inhibits IL-6. In some embodiments, the IL-6 inhibitor directly inhibits IL-6 and is selected from the group consisting of a small molecule, a peptide, an antibody or a fragment thereof and a nucleic acid molecule. In another embodiment, the IL-6 inhibitor indirectly inhibits IL-6 via IL-6 receptor (IL-6R) wherein inhibitor of IL-6R is selected from the group consisting of a small molecule, a peptide, an antibody or a fragment thereof and a nucleic acid molecule. In some embodiments, the nucleic acid molecule is a siRNA molecule specific for IL-6 or IL-6R. In some embodiments, the the antibody is selected from the group consisting of monoclonal antibody or fragment thereof, a polyclonal antibody or a fragment thereof, chimeric antibodies, humanized antibodies, human antibodies, and a single chain antibody. The pharmaceutical compositions according to the invention can contain any pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral or enteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection. Methods for these administrations are known to one skilled in the art.

The pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Before administration to patients, formulants may be added to the IL-6 inhibitor. A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

In some embodiments, polymers as formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

Another drug delivery system for increasing circulatory half-life is the liposome. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

### Kits

In various embodiments, the present invention provides a kit for treating or inhibiting antibody mediated transplant rejection. The kit is an assemblage of materials or components, including a direct or indirect inhibitor of IL-6. Thus in some embodiments, the kit contains a composition including IL-6R inhibitor, Tocilizumab.

The exact nature of the components configured in the inventive kit depends on its intended purpose. In one embodiment, the kit is configured particularly for human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to treat or inhibit ABMR in a subject. Optionally, the kit also contains other useful components, such as, measuring tools, diluents, buffers, pharmaceutically acceptable carriers, syringes or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a bottle used to contain suitable quantities of an inventive composition containing a direct or indirect inhibitor of IL-6, such as Tocilizumab (an IL-6R inhibitor). The packaging material generally has an external label which indicates the contents and/or purpose of the kit and/or its components.

### EXAMPLES

The following examples are not intended to limit the scope of the claims to the invention, but are rather intended to be exemplary of certain embodiments. Any variations in the exemplified methods which occur to the skilled artisan are intended to fall within the scope of the present invention.

### Example 1

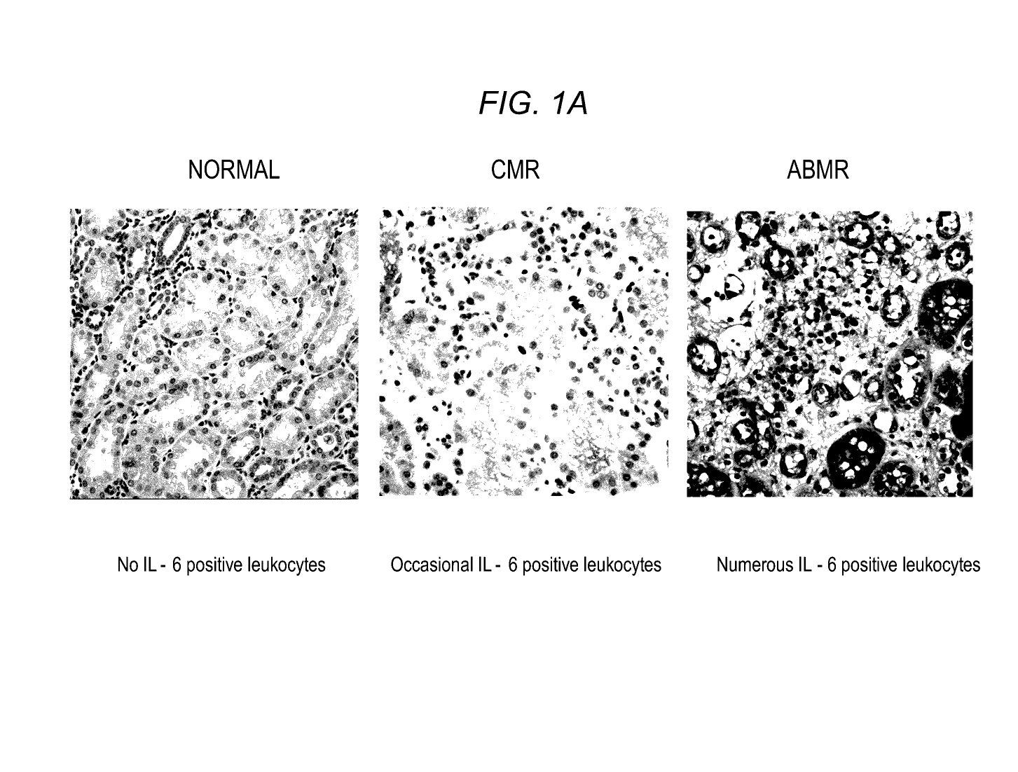

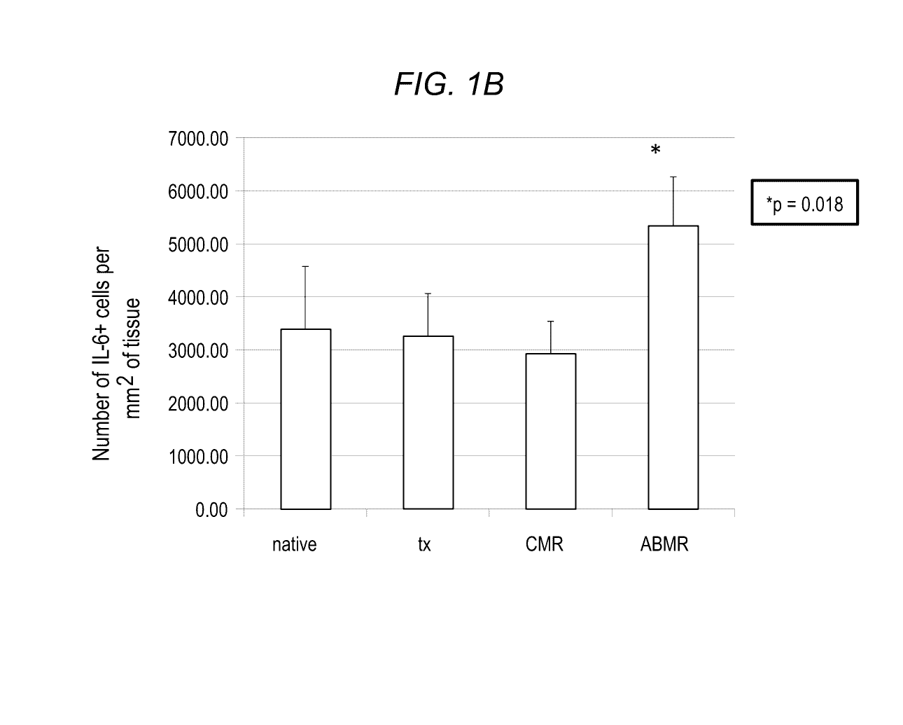

As shown in FIGS. 1A and 1B IL-6 might play an important role in antibody-mediated injury to allografts. FIG. 1A shows representative staining of normal kidney tissue, tissue from a patient with cellular rejection and a biopsy from a patient with antibody-mediated rejection. In this instance, there are numerous IL-6+ cells in the biopsy of ABMR compared with CMR and normal tissue. FIG. 1B shows data from a larger analysis of ABMR biopsies compared to other diagnoses. Using morphometric scanning analysis, we were able to show a significant increase in IL-6 expression in biopsies with ABMR.

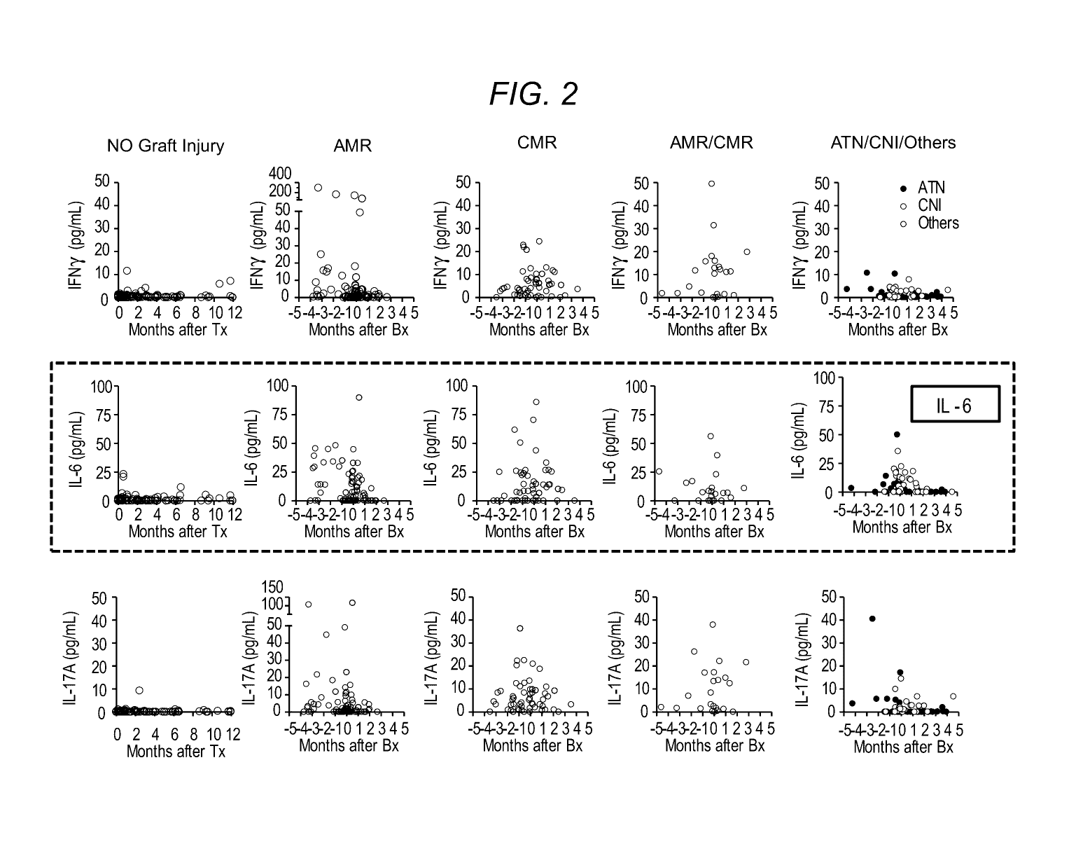

As shown in FIG. 2, cytokine levels were low in most patients who did not have biopsies post-transplant. In contrast, the cytokine levels pre-, at and post-Bx were elevated in many patients who had Bx (AMR, CMR, ATN, CNI toxicity). Interestingly, these cytokine levels quickly became fairly low levels after the treatment of AMR, while the levels remained high after the treatment of CMR.

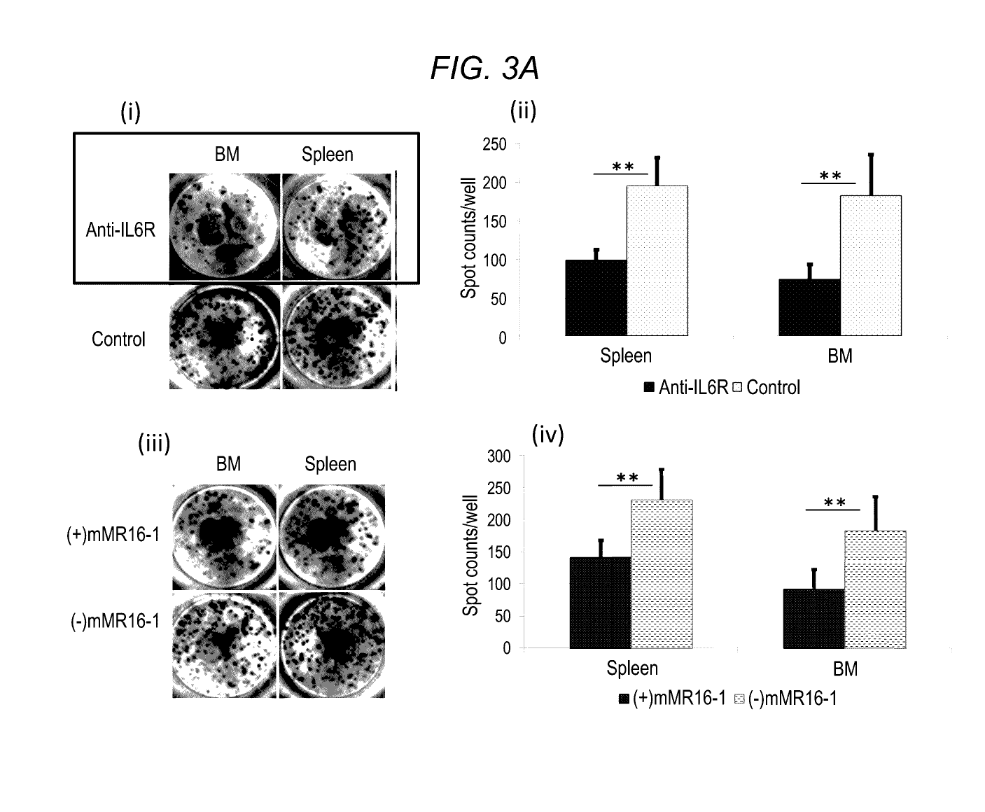

As shown in FIG. 3A and FIG. 3B, data from our group using a mousenized anti-IL-6R antibody in a mouse model of allo-sensitization. Briefly, after sensitization, we can detect anti-HLA-A2 antibodies in sera of animals. Anti-IL-6R significantly reduces circulating levels of anti-HLA-A2 antibodies. In addition, there is a significant reduction of antibody producing plasma cells isolated from the bone marrow and spleens of sensitized animals (FIG. 3A). Further, plasma cells producing anti-HLA-A2 antibody in the bone marrow are inhibited by anti-IL-6 therapy (FIG. 3B). Addition of anti-IL-6 treatment of conditioned media of bone marrow plasma cells from untreated sensitized animals also significantly reduced anti-HLA-A2 antibody production.

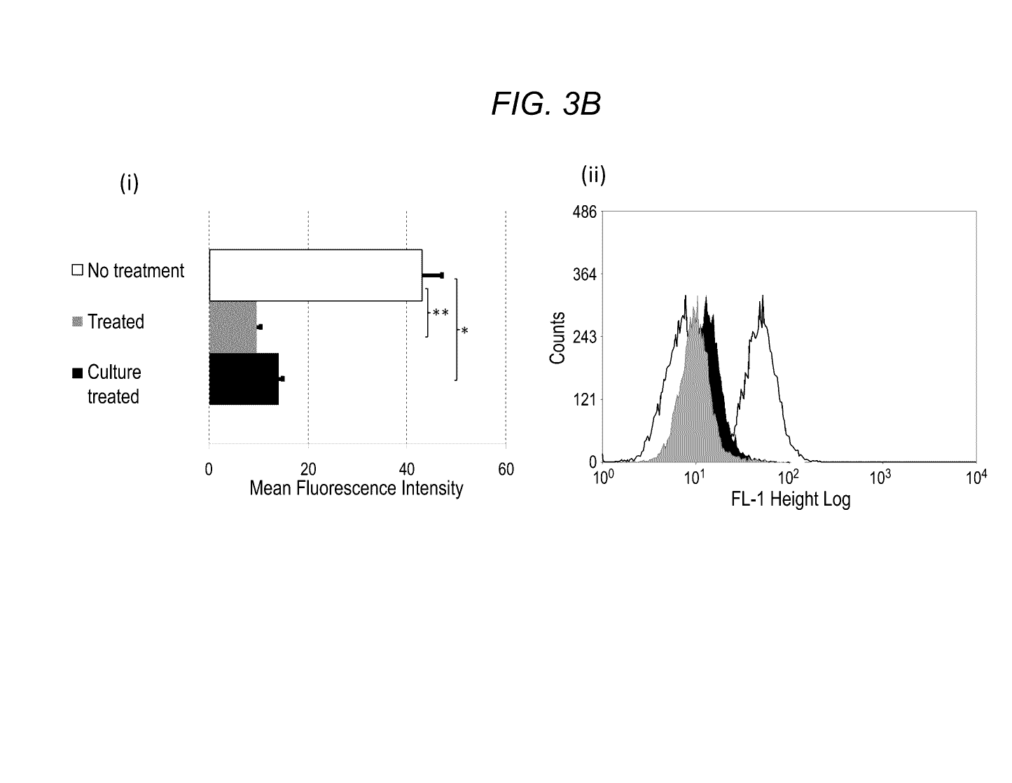

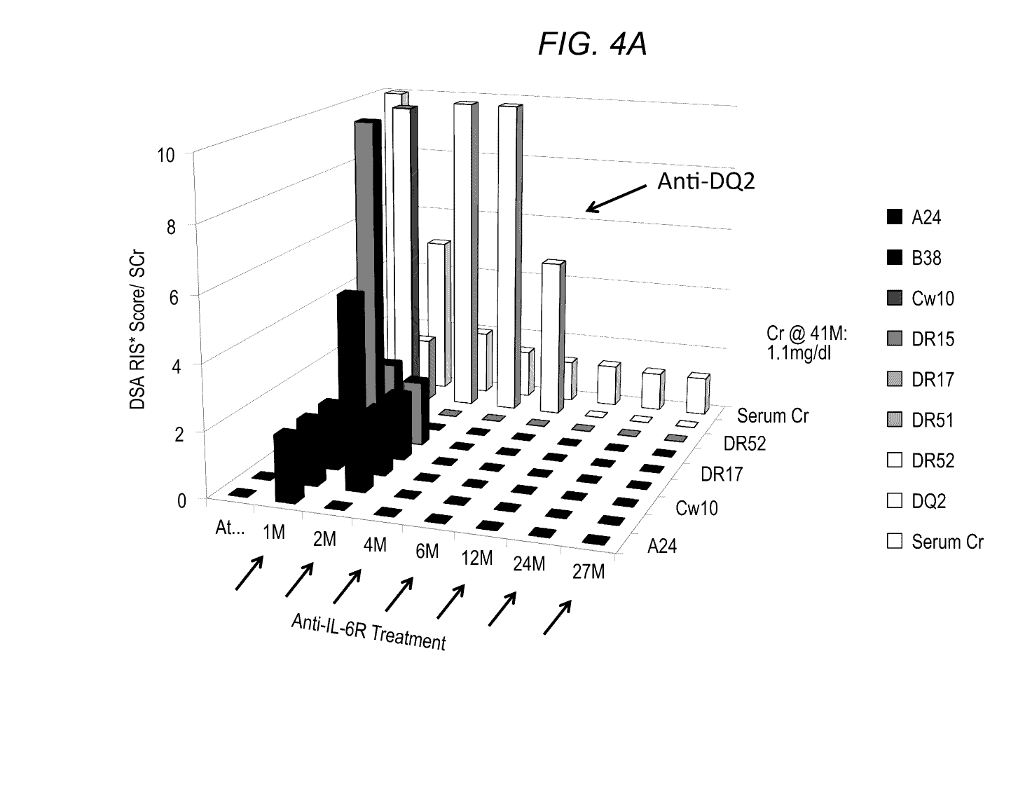

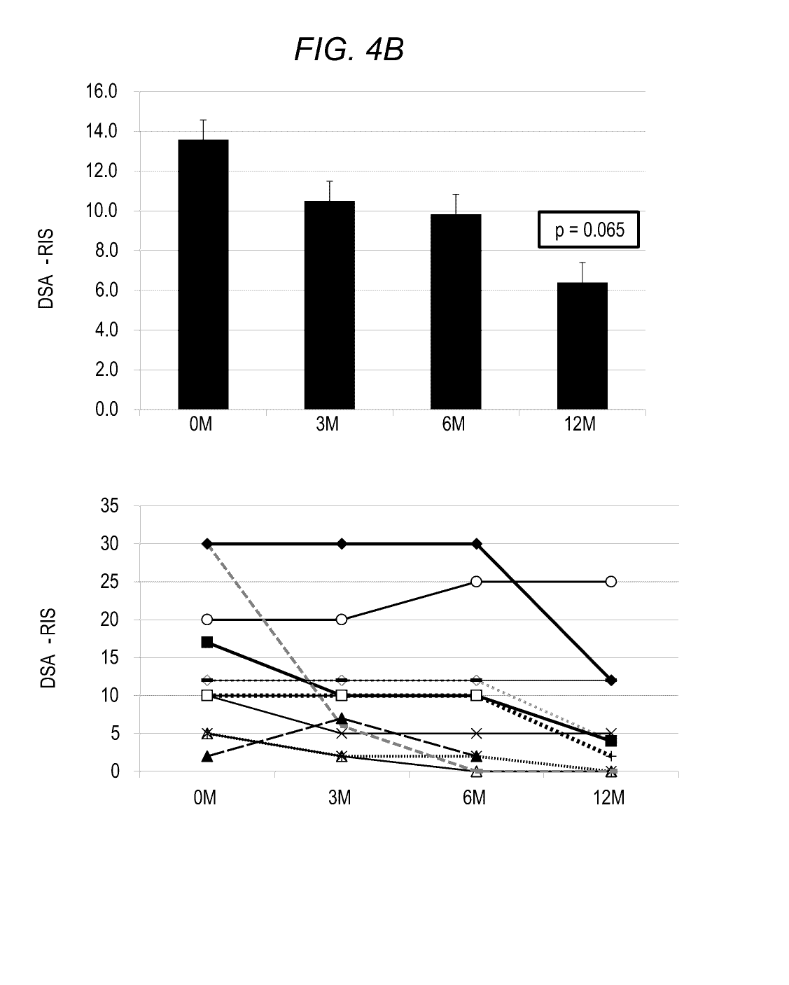

FIG. 4A shows the clinical and DSA course of the 1^{st} patient treated for ABMR. Briefly, the patient developed severe ABMR with multiple DSA elevations post-transplant. After PLEX+IVIG showed no improvement, the patient received Tocilizumab 8 mg/kg×5 doses. The patient responded well to the initial dose with increasing urine output and declining serum creatinine level. After 41 M (last follow up) patient has no DSAs and a serum creatinine 1.1 mg/dl. FIG. 4B shows donor specific antibody RIS score post Tocilizumab treatment.

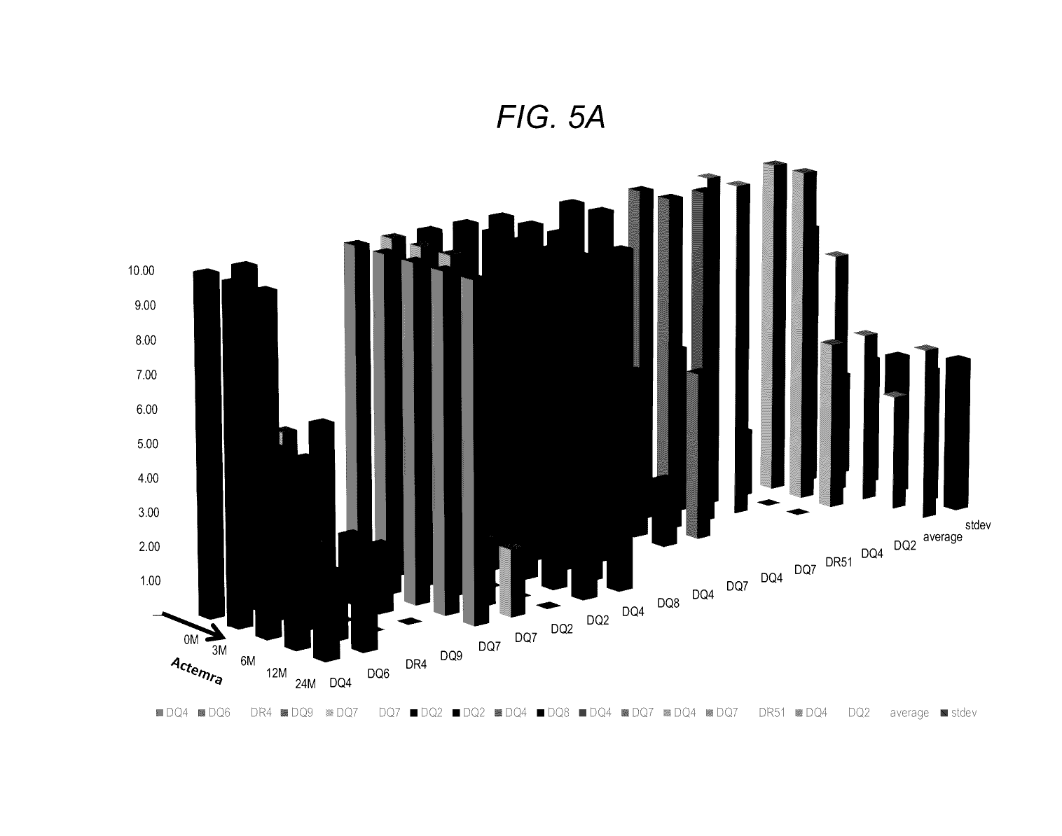

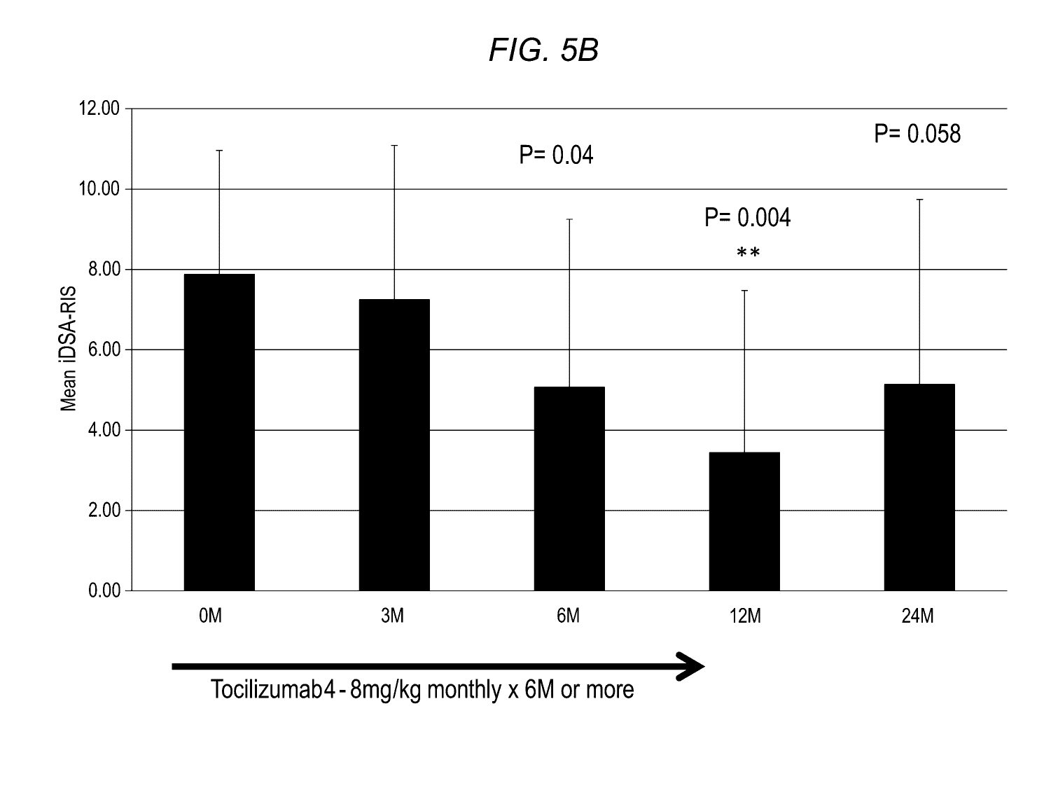

FIG. 5A shows immunodominant DSA Scores for ABMR Patients Treated with TCZ (n=18) *Relative Intensity Scale (RIS) [0 points=No DSA; 2 points=<5000 MFI {weak}; 5 points=5000-10,000 MFI {moderate}; 10 points=>10,000 MFI {strong}]. FIG. 5B shows the mean immunodominant DSA values for TCZ treated patients.

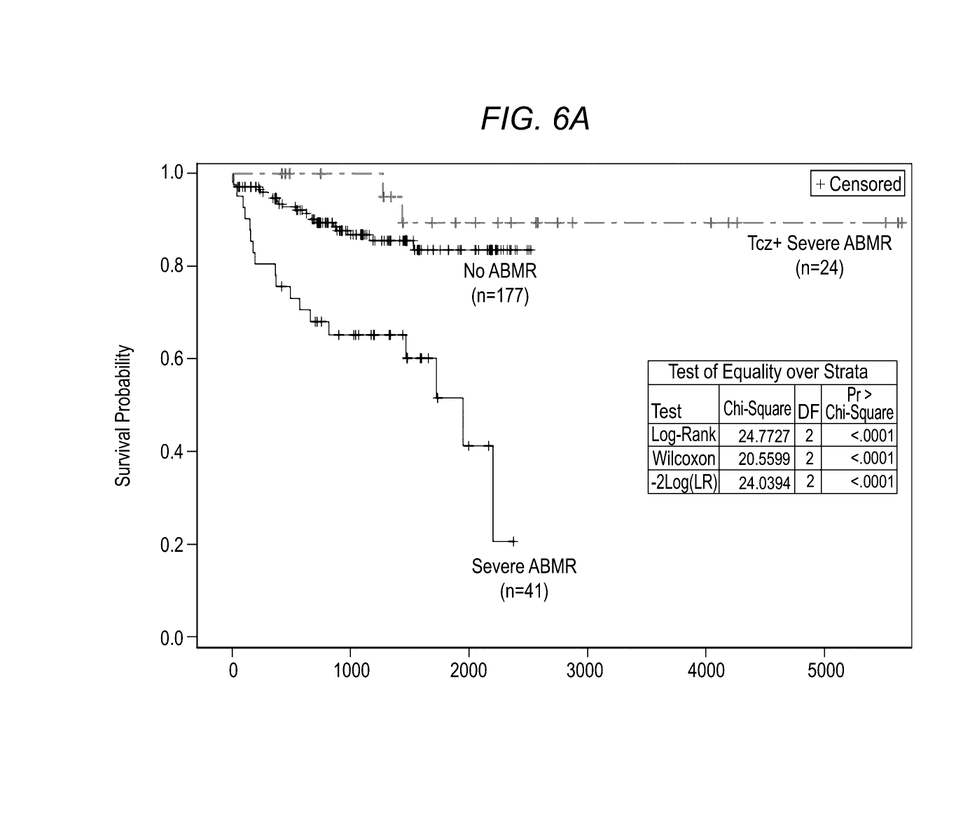

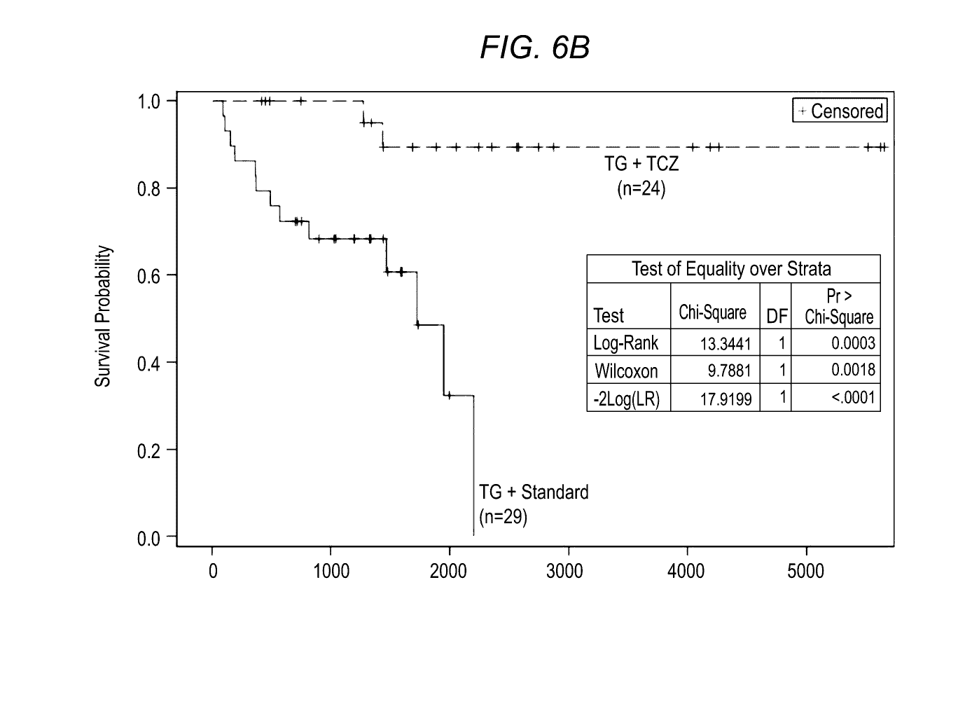

As shown in FIG. 6A, Tocilizumab significantly improves graft survival in patients with severe ABMR compared to standard-of-care treatment group (p<0.0001). These findings contrast to the known poor outcomes for patients with this condition treated with current standard-of-care. FIG. 6B shows the outcomes in patients with ABMR and transplant glomerulopathy (TG). The curves compare patients with TG treated with standard-of-care compared to those who received 6-12 M of Tocilizumab treatment. TG is usually a devastating complication of kidney transplantation with loss of allograft function over months to years. Here, we see that Tocilizumab was able to stabilize renal function in these patients, and to date, prevent their return to dialysis. No deaths were seen in the Tocilizumab group.

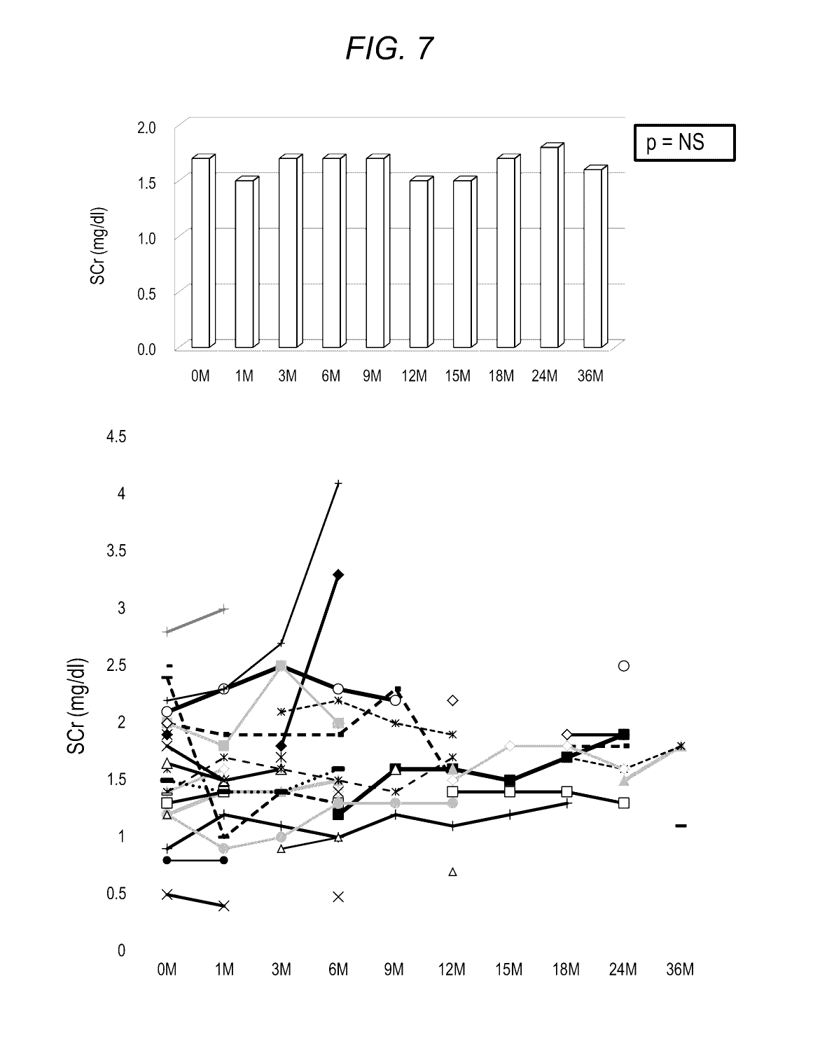

FIG. 7 shows the average serum Creatinine (mg/dl) post-Tocilizumab treatment. Creatinine levels assess kidney function.

### Example 2

ABMR is a serious and significant complication of transplantation after desensitization (DES) in highly-HLA sensitized patients (HS). Therapies for treatment of ABMR usually include IVIG+Rituximab (I+R) and/or PLEX+I+R. Despite these treatments, many ABMR+ patients experience irreversible allograft injury and progress to ESRD with return to dialysis. The personal and economic costs of allograft failure are enormous. Recent data suggests an important role for IL-6 in mediation of ABMR. Data from animal models show a significant modification of allo-antibody responses and chronic ABMR, a reduction in CD138+ plasma cells and induction of Tregs using anti-IL-6R therapy. Here, we examined the utility of a novel agent (Tocilizumab {TCZ} {anti-IL-6R}, {Genentech-Roche}) for treatment of ABMR+/DSA+ patients resistant to treatment with I+R or PLEX+I+R.

We identified 11 patients who developed ABMR+ including patients with chronic TG and DSA+. Detailed characteristic of each patient is shown in FIG. 1. Briefly, patients received TCZ @ 8 mg/kg, monthly×3-9M. Patients were monitored for DSA levels and renal function.

All patients had significant pathologic findings of ABMR, usually with elevated DSA levels at time of treatment. After TCZ treatment, DSA levels decreased in 6/11 patients (55%) while 3/11 (27%) remained stable over the 12M observation period. One patient experienced CMV and PCP infection and recovered, otherwise, no SAEs were observed. Renal function was stable in 10/11 patients (91%) while one patient lost the allograft due to unremitting ABMR+. One patient with nephrogenic systemic fibrosis showed significant improvement in skin softening and ROM of L hand post-TCZ. [see Table 1]

From this small case series, we are cautiously optimistic that TCZ may offer a new option for dealing with severe PLEX+I+R resistant ABMR+ patients. Potential benefits of TCZ therapy include inhibition of B-cell activation, reduction in plasma cell Ig production and induction of Tregs.

TABLE 1
Use of Tocilizumab (TCZ) in DSA+ ABMR Patients Resistant to IVIG + Ritux (I + R).

| | 1 | 2 | 3 | 4 | 5 | 6 | 7 | 8 | 9 | 10 | 11 |
|---|---|---|---|---|---|---|---|---|---|---|---|
| M/F | F | F | F | F | F | M | F | M | F | M | M |
| Age | 51 | 40 | 41 | 30 | 69 | 37 | 59 | 38 | 67 | 17 | 17 |
| Transplant Type | DD | DD | DD | LD | DD | DD | DD | LD | DD | LD | DD |
| (DD/LD) | | | | | | | | | | | |
| Time from transplant | 71 | 67.5 | 53.3 | 42.7 | 13.4 | 6.1 | 3.8 | 161.1 | <1 | 159.3 | 22 |
| to treatment (M) | | | | | | | | | | | |
| DSA RIS* | | | | | | | | | | | |
| @Baseline | 12 | 17 | 5 | 10 | 5 | 20 | 0 | 10 | 28 | 12 | 30 |
| @6 M post treatment | 12 | 10 | 0 | N/A | 2 | 30 | 0 | 10 | 0 | 12 | 30 |
| @12 M post treatment | 9 | 4 | N/A | 5 | 0 | N/A | N/A | N/A | 0 | 12 | 30 |
| # of Actemra Doses | 6 | 6 | 6 | 3 | 9** | 6 | 6 | 6 | 5 | 4 | 4 |
| (dose: 8 mg/kg monthly) | | | | | | | | | | | |
| SCr(mg/dl)/GFR | | | | | | | | | | | |
| (ml/min) | | | | | | | | | | | |
| @Baseline | 2/26 | 1.3/45 | 1.2/50 | 4.2/12 | 1.64/39 | 2.1/36 | 0.9/64 | 2/38 | 2.4/20 | 1.4/67 | 1.3/72.7 |
| @6 M post treatment | 2/26 | 1.2/50 | 1.5/38 | 3.9/14 | 2.24/27 | 2.2/34 | 1/57 | 1.9/40 | 1.3/41 | 1.4/67 | 1.3/72.7 |
| @12 M post treatment | 2.2/23.5 | 1.6/36 | 1.6/36 | LostGraft | 1.9/32 | N/A | 1.2/46 | 1.5/52 | 1.2/45 | N/A | N/A |
| Serious | No | No | No | No | Yes | No | No | Yes | No | No | No |
| Infection | | | | | (e. coli | | | (CMV/PCP) | | | |
| | | | | | no) | | | | | | |

*RIS: Relative Intensity Score (10 points = strong dsa, 5 points = moderate dsa, 2 points = weak dsa).
**Patient received a dose of 4 mg/kg instead of 8 mg/kg.

### Example 3

Limited options are available for treatment of ABMR in highly-HLA sensitized patients (HS). Treatment options include FR, PLEX+I+/−R, C5-inhibitor & bortezomib. Approximately 25% of HS patients are at risk for ABMR+ with development of chronic TG & return to dialysis. Emerging data suggest IL-6 may have an important role in ABMR+ injury & inflammation that occurs post-tx. Here, we examined the long term use of TCZ for treatment of ABMR+/DSA+ (+/−angiotensin-1 receptor (AT1R) antibody (ab) patients unresponsive to treatment with I+R or PLEX+I+R.

We identified 23 ABMR+ patients including those with chronic TG, DSA+ and/or AT1R ab+. Briefly, patients received TCZ at 4-8 mg/kg, monthly×3-11M. Patients were monitored for DSA and renal function. AT1R ab results were also monitored in 7/23 (30%) patients.

21/23 (91%) patients had significant pathologic findings of ABMR (17/21 (81%) patients with TG) usually with elevated DSA levels and/or elevated AT1R ab at time of treatment. After TCZ treatment, DSA levels decreased in 10/23 patients (43%) while 6/23 (26%) remained stable at 18M. One patient had significant rebound after TCZ was discontinued. 4/6 (67%) patients with stable DSA scores had C1Q positive DSAs, 3 patient had both C1Q+ DSA & AT1R ab+ & 1 TG patient had no identifiable antibodies. SAEs included, 2 patients had TCZ postponed after 4th dose d/t temporary vision loss & eye numbness, 1 patient had dose adjusted for neutropenia. Renal function was stable in 21/23 patients (91%) while one patient lost the allograft due to unremitting ABMR+. One patient with NSF showed significant improvement in skin softening with stable Cr & improved DSA-RIS. [see Table 2]

From our single center experience, TCZ continues to show promising results in ABMR+ patients resistant to PLEX+/−I+R. Potential benefits of TCZ therapy include inhibition of B-cell activation, reduction in plasma cell Ig production and induction of Tregs.

TABLE 2
TCZ patients' outcomes.

| | | TCZ Treated Patients | |
|---|---|---|---|
| | | (n = 23) | p-value |
| Mean DSA RIS* | @ Baseline | 12.4 ± 7.9 | — |
| | @ 3 M | 9.5 ± 7.4 | 0.2984 |
| | @ 6 M | 7.6 ± 8.1 | 0.1031 |
| | @ 12 M | 7.1 ± 6.6 | 0.064 |
| | @ 18 M | 8.7 ± 11.2 | 0.375 |
| Renal Function | @ Baseline | 1.6 ± 0.5 | 0.822 |
| (SCr in mg/dl) | @ 3 M | 1.6 ± 0.6 | 0.518 |
| | @ 6 M | 1.8 ± 0.8 | 0.358 |
| | @ 12 M | 1.6 ± 0.4 | 0.862 |
| | @ 18 M | 1.6 ± 0.3 | 0.810 |
| | @ 24 M | 1.7 ± 0.5 | 0.597 |

| AT1R ab (# patients, U/ml) | | Total patients w/ |
|---|---|---|
| | | results: 7/23 (30%) |
| | Strong (≧17) | 5/7 (71.4%) |
| | Intermediate (11-16) | 1/7 (14.3%) |
| | Negative (≦10) | 1/7 (14.3%) |
| | NA | 16/23 (70%) |
| C1Q + DSA (# patients) | | Total patients w/ |
| | | results: 14/23 (61%) |
| | Yes | 11/14 (79%) |
| | No | 3/14 (21%) |
| | NA | 9/23 (39%) |

| | |
|---|---|
| Both AT1R ab+ and C1Q+ (# patients) | 3/23 (1.3%) |
| Graft Loss/Death | 2 (1%)/0 (0%) |
| #Patients with Baseline Biopsies | 21/23 (91%) |
| % TG biopsies | 17/21 (81%) |

*Relative Intensity Scale (RIS) [0 points = No DSA; 2 points = <5,000 MFI; 5 points = 5,000-10,000 MFI; 10 points = >10,000 MFI]

### Example 4

Background: Extending the function and longevity of renal allografts is the primary goal of transplant medicine. Development of donor-specific antibodies (DSAs) post-transplant leads to chronic antibody-mediated rejection (CABMR) and transplant glomerulopathy (TG) which accounts for the majority of graft losses in the U.S. This results in increased cost to the health care system and reduced length and quality of life for affected patients. Currently, there are no approved therapies for treatment of CABMR. Mounting evidence suggests the pro-inflammatory cytokine, interleukin 6 (IL-6) plays an important role in DSA generation and ABMR. Methods: Over the past 5 years, we identified 36 renal transplant patients who presented with DSA+ CABMR and TG. These patients had failed standard of care (SOC) treatment with IVIG+rituximab and were offered rescue therapy with the anti-IL-6 receptor monoclonal, tocilizumab. Patients were treated for 6-18 months with monthly injections of 8 mg/kg and were monitored for graft and patient survival, renal function, adverse events and changes in DSAs for up to 5 years. Outcomes were compared to a retrospective cohort of 39 CABMR and TG patients treated with SOC. Results: Patients treated with tocilizumab demonstrated superior graft survival and a tendency for a better patient survival (p=0.0004 and p=0.1256), respectively. Reductions in DSAs and stabilization of renal function were also seen. No significant AEs/SAEs were observed. Anti-IL-6R therapy provides superior long-term outcomes for patients with CABMR and TG with limited side effects. Inhibition of the IL-6/IL-6R pathway may represent a novel approach to improve allograft function and extend patient lives.

### Patients and Treatment Design

This single center, open label protocol was developed and conducted at Cedars-Sinai Medical Center. This was not a sponsored trial. Between 2011 and 2016, 36 patients were offered treatment with tocilizumab (8 mg/kg monthly for 6-18M). The objectives of this treatment were to provide a “rescue” therapy for patients with CABMR and TG who had failed treatment with IVIG+rituximab+/−PLEX as standard of care (SOC). These patients were deemed treatment failures if they failed to show improvements in serum creatinines and/or reductions in DSA levels three months after SOC treatment. Eligible patients were 6-69 years, all of whom had kidney allograft biopsies with evidence of ABMR, CABMR and usually TG with DSA positivity. Patients received tocilizumab monthly for 6-18 months (M) with monitoring of renal function, AE/SAE, graft and patient survival and DSA levels. Nine patients also underwent repeat biopsies 1 year post-tocilizumab treatment to assess impact of therapy on pathological features of CABMR. Outcomes in tocilizumab treated patients were compared to a retrospective cohort of 39 kidney transplant patients with biopsy proven CABMR and TG who were DSA positive and exhibited similar demographics, but received treatment with IVIG+rituximab+/−PLEX as standard of care.

### Study Assessment

Prior to initiation of tocilizumab, all patients were tested for exposure to tuberculosis using PPD skin test. After initiation of therapy, patients remained on their standard immunosuppression and were monitored for viral infections (CMV, EBV and polyoma BK) every 3 months using PCR techniques. Infusions were given in our infusion center with patients being constantly monitored for AE/SAEs related to infusions. Doses of tocilizumab were given at 4-8 mg/kg over 30 minutes after pre-medication with steroids, acetaminophen and Benadryl. Patients were also monitored for hypogammaglobulinemia (Total IgG <750 mg/ml) and if present, were treated with IVIg infusions monthly (1 gm/kg initially, then 0.5 gm/kg monthly for 6 months). Renal function was assessed monthly and DSAs every 3 months. Nine patients also had repeat biopsies at 1 year post-tocilizumab. Observations were carried out for up to 5 years with final assessments of patient and graft survivals since initial biopsy and compared to the retrospective cohort.

### Statistical Analysis

Continuous variables are described using means and standard deviations (SDs) or median and Interquartile ranges (IQR). We compared means and proportions between groups using Mann-Whitney test and Fisher's exact test. The kidney survival analysis was performed from the time of ABMR until a maximum follow-up of 6 years with kidney graft loss as the event of interest, defined as the patient's return to dialysis. For the 2 (2.7%) patients who died with a functioning graft, graft survival was censored at the time of death. Kidney allograft survival according to the Standard of care and Tocilizumab treatment was plotted using Kaplan-Meier curves and compared using the log-rank test.

Because our study has a limited sample size (n=75), we adopted a Random Forest Survival (RFS) analysis to identify prognostic factors for the graft survival. RSF is known to have good performance for small sample size and many potential predictors. In some situations RSF is known to outperform the traditional stepwise Cox model. RSF is a non-parametric machine learning method developed by Ishwaran et al, which is an extension of Random Forest to right-censored survival settings (Ishwaran H, et al. (2008). Random Survival Forests.” The Annals of Applied Statistics, 2(3), 841). Our RSF analysis was generated by creating 2000 trees; each tree was developed using a bootstrap sample of the original dataset. At each branch, a random set of variables are chosen as candidates to split the branch into 2 other branches, and the variable maximizing the log-rank statistic using 3 randomly selected split points was used for splitting. Splitting of branches to create the tree continues as long as possible until terminal branches have no fewer than 3 events (Segal et al. Regression trees for censored-data. Biometrics. 1988; 44:35-47). Prognostic factors for graft loss were studied by means of the minimal depth rule (Ishwaran H, et al. 2010 High Dimensional Variable Selection for Survival Data.” J. Amer. Statist. Assoc., 105, 205-217). The minimal depth for a splitting variable evaluates the minimal distance between that variable relative to the root node. The most predictive variables for the cohort are defined as those whose minimal depth (average aver the forest) is smaller than the mean minimal depth determined under the null hypothesis of no effect. In addition we calculated variable importance (VIMP), which measures the increase in prediction error when the variable is “noised-up”. A variable is noised-up by random permitting its values (Ishwaran H (2007). Variable Importance in Binary Regression Trees and Forests.” Electronic Journal of Statistics, 1, 519{537). A positive value indicates a predictive covariate. The C-index (Pencina M J, D'Agostino R B. (2004). Overall C as a measure of discrimination in survival analysis: model specific population value and confidence interval estimation. Statis Med, 23: 2109-2123) was calculated using out-of-bag (OOB) data. A value of 1 for C-index corresponds to perfect prediction, while a value of 0.5 does not perform better than random prediction. RSF was performed using the “rfsrc” function from “randomForestSRC” in R.

We used STATA (version 14, Data Analysis and Statistical Software) and R (version 3.2.1, R Foundation for Statistical Computing) for the descriptive and survival analyses. All the statistical tests were 2-sided, and probability values <0.05 were considered significant.

### Baseline Characteristics of the Kidney-Allograft Recipients

In total 75 patients were included in the main analysis, in which 36 received the Tocilizumab (48%) and 39 received the SOC treatment (52%). The baseline study characteristics at the time of biopsy for the tocilizumab and SOC groups are shown in Table 3. Briefly, the two groups were similar except for a larger number of re-transplant patients (n=28 (71.79%) in the SOC group vs 17 (47.22%) in the Tocilizumab group, p=0.026), higher number of HLA class I DSAs (1.70±1.41 in the SOC group vs 0.38±0.65 in the Tocilizumab group, p<0.001) and lower eGFR (30.39±19.56 mL/min/1.73 m² in the SOC group vs 49.08±35.44 mL/min/1.73 m² in the Tocilizumab group, p=0.002) at the time of biopsy in the SOC group. There were significantly more graft losses in the SOC group (n=19 (48.72%) vs n=4 (11.11%), p<0.001) as well.

TABLE 3
Characteristics of patients according to the treatment: Standard of care vs Tocilizumab.

| | | | |
|---|---|---|---|
| All patients | Standard of care | Tocilizumab | |
| (n = 75) | (n = 39) | (n = 36) | |
| n | n | n | P^{a} |

Recipient characteristics

| | | | | | | | |
|---|---|---|---|---|---|---|---|
| Age (years), mean (SD), y | 75 | 43.25 (15.39) | 39 | 40.84 (11.76) | 36 | 45.86 (16.64) | 0.058 |
| Gender male, No. (%) | 73 | 40 (54.79) | 39 | 21 (53.85) | 34 | 19 (55.88) | 0.525 |
| Graft rank > 1, No. (%) | 75 | 45 (60.00) | 39 | 28 (71.79) | 36 | 17 (47.22) | 0.026 |

Donor characteristics

| | | | | | | | |
|---|---|---|---|---|---|---|---|
| Deceased donor, No. (%) | 75 | 48 (64.00) | 39 | 28 (71.79) | 36 | 20 (55.56) | 0.111 |
| ECD donor, No (%) | 75 | 2 (2.67) | 39 | 1 (2.56) | 36 | 1 (2.78) | 0.733 |
| Cold ischemia Time > 24 h, No (%) | 75 | 10 (13.33) | 39 | 8 (20.51) | 36 | 2 (5.56) | 0.057 |
| Delayed graft function^{b}, No (%) | 75 | 17 (22.67) | 39 | 11 (28.21) | 36 | 6 (16.67) | 0.180 |

Immunology at the time of ABMR

| | | | | | | | |
|---|---|---|---|---|---|---|---|
| HLA mismatches, mean (SD) | 67 | 4.28 (1.52) | 39 | 4.44 (1.55) | 28 | 4.07 (1.46) | 0.261 |
| Anti-HLA DSA positive, No (%) | 71 | 63 (88.73) | 37 | 32 (86.49) | 34 | 31 (91.18) | 0.404 |
| Number of anti-HLA DSA, mean (SD) | 71 | 2.56 (2.12) | 37 | 3.43 (2.44) | 34 | 1.62 (1.13) | <0.001 |
| Number of Class I, mean (SD) | 71 | 1.07 (1.29) | 37 | 1.70 (1.41) | 34 | 0.38 (0.65) | <0.001 |
| Number of Class II, mean (SD) | 71 | 1.44 (1.28) | 37 | 1.64 (1.60) | 34 | 1.24 (0.78) | 0.678 |

Renal function at the time of ABMR

| | | | | | | | |
|---|---|---|---|---|---|---|---|
| eGFR, mean (SD), mL/min/1.73 m² | 73 | 39.09 (29.42) | 39 | 30.39 (19.56) | 34 | 49.08 (35.44) | 0.002 |
| Follow-up (years), mean (SD), y | 75 | 2.81 (2.00) | 39 | 2.39 (1.91) | 36 | 3.26 (2.04) | 0.075 |
| Graft loss, No. (%) | 75 | 23 (30.67) | 39 | 19 (48.72) | 36 | 4 (11.11) | <0.001 |

Abbreviations: DSA: donor-specific anti-HLA antibodies;
HLA: human leucocyte antigen;
eGFR: estimated Glomerular filtration rate;
SD: Standard deviation
^{a}Fisher exact tests were conducted to compare proportions, and Mann witney tests were conducted to compare continuous variables.
^{b}Delayed graft function was defined as the use of dialysis in the first postoperative week.

### Long-Term Kidney and Patient Survival, Injury Phenotypes and Function

Overall, the median follow-up after ABMR was 2.46 years (IQR: 1.46-3.60). Overall, the one, three and six-year allograft survival rates were 85, 72 and 56%, respectively. The median follow-up times were 2.51 years (1.82-3.81) for the patients treated by Tocilizumab and 1.97 years (0.40-3.59) for the patients treated by SOC treatment.

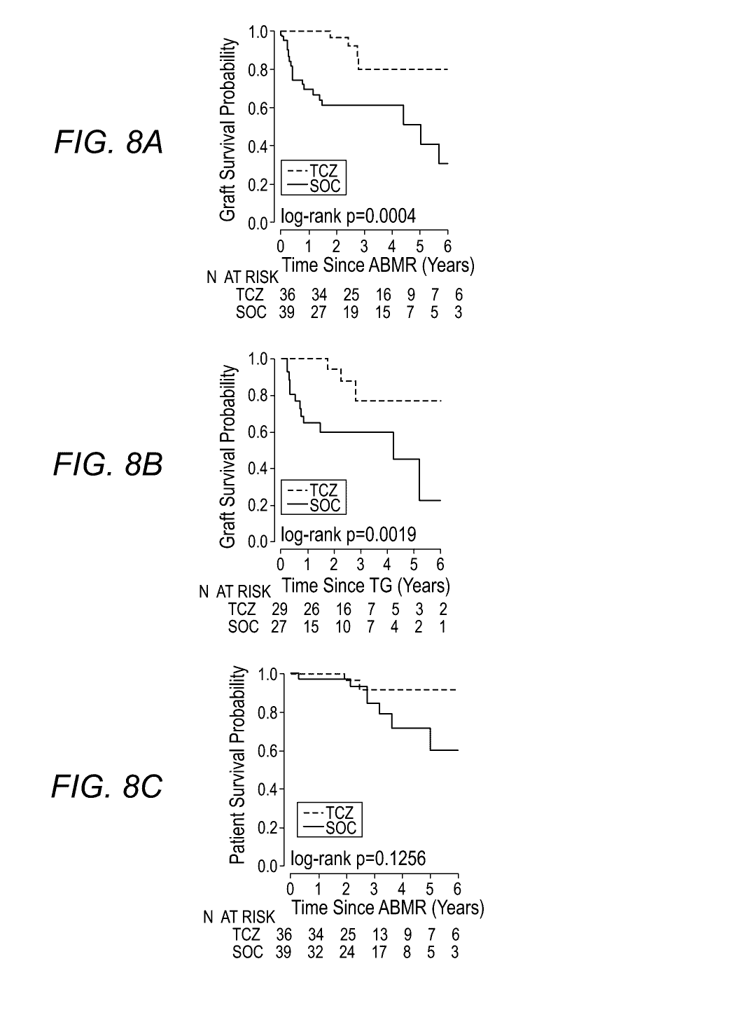

FIG. 8A-C shows the Kaplan-Meyer assessments of graft and patient survival according to the treatment (Tocilizumab vs SOC). FIG. 8A demonstrates a superior graft survival for Tocilizumab treated patients over the study period since initial biopsy. The patients treated with Tocilizumab exhibited a better allograft survival (80% at 6 years post-ABMR) than the patients treated by SOC (31% at 6 years post-ABMR, log-rank test, p=0.0004). After TG, the patients treated by Tocilizumab also exhibited a better allograft survival (82% at 6 years post-ABMR) than the patients treated by SOC (17% at 6 years post-ABMR, log-rank test, p=0.0010) (FIG. 8B). Patient survival is presented in FIG. 8C. Patients in the SOC group had a tendency to higher death rates over the study period (60% at 6 years post-ABMR) compared to the Tocilizumab group (91% at 6 years post-ABMR, log-rank test, p=0.1256).

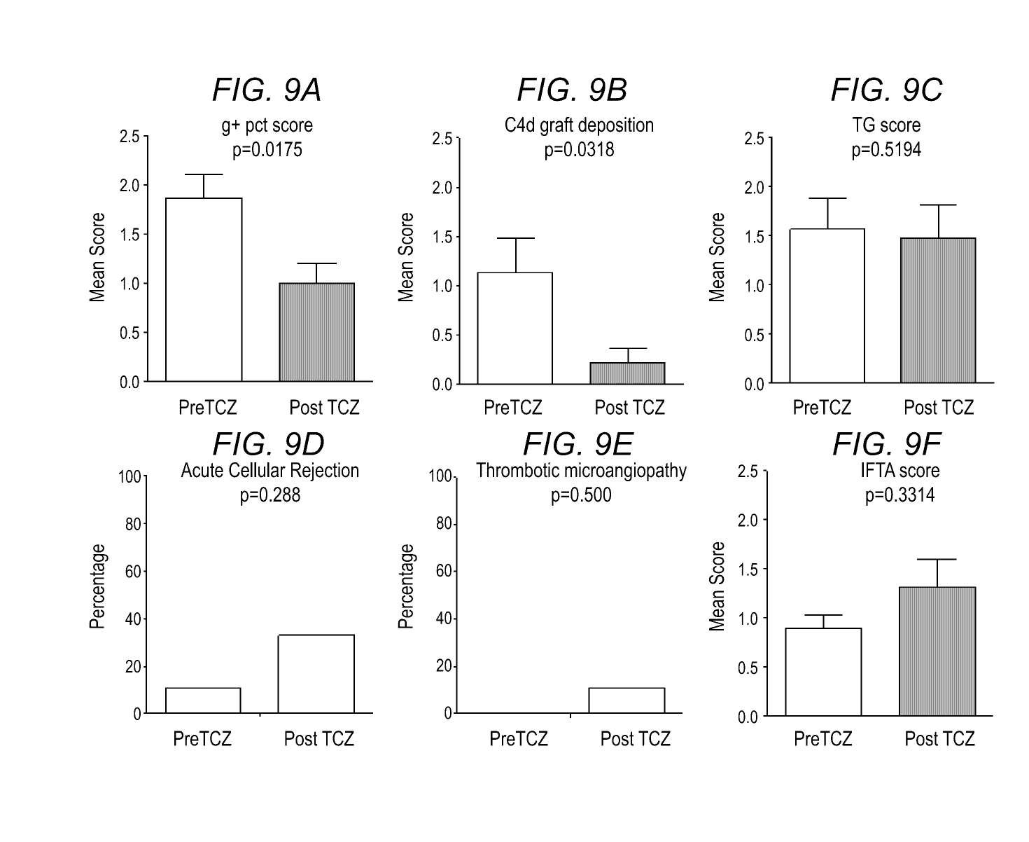

Determination of eGFR values showed that the eGFR values for tocilizumab treated patients over the study period remained stable. FIG. 9A shows the impact of tocilizumab on immunodominant (iDSA) levels. iDSAs are defined as the strongest DSA detected in the patients' sera. FIG. 9B shows the Banff 13 scoring for ABMR on pre- and post-tocilizumab (1 year) biopsies from 9 patients. Significant reductions in C4d+ (p=0.0318) deposition and g+ptc scores (p=0.0175) were seen post-tocilizumab treatment.

### Determinants of Kidney Allograft Loss

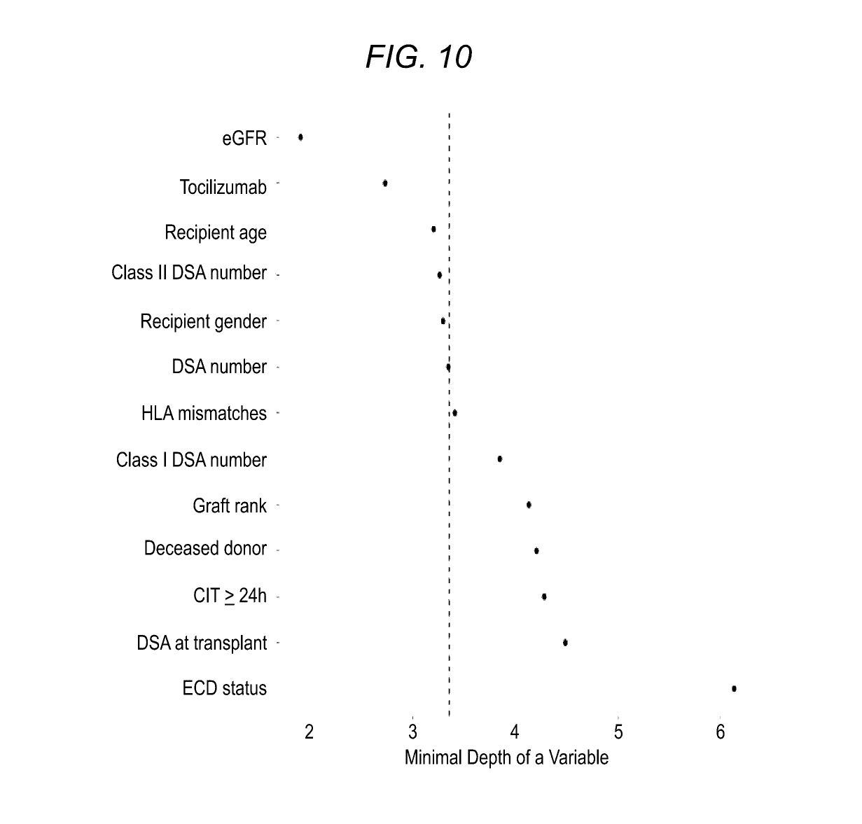

We used a Random Forest Survival (RFS) analysis to identify prognostic factors for the graft survival. The RFS analysis was generated by creating 2000 trees. Using the minimal depth measure, the 2 most important variables were the eGFR and the treatment by Tocilizumab following by the recipient's age, the number of class II DSA, the recipient gender, and the overall number of DSA. (FIG. 10). The model discrimination using OOB Harrell's C index was 0.77.

We next assessed AEs/SAEs associated with tocilizumab administration. Briefly 3 patients had the following: transient visual disturbance after the 3^{rd} dose of TCZ but resolved soon after, trichodysplasia spinulosa (benign skin condition related to polyoma virus) 1 month after completion of 12^{th} dose but resolved after decreasing immunosuppression, 1 patient had fever after the 2^{nd} TCZ dose and when fever resolved TCZ resumed and patient had no issues after. A total of 4 patients had hypogammaglobulinemia (serum Immunoglobulin G<751 mg/dl) however 2 of the 4 patients had hypogammaglobulinemia before TCZ started. The other 2 patients: 1 patient developed hypogammaglobulinemia after the 2^{nd} TCZ dose and another patient after the 6^{th} TCZ dose. A total of 2 patients expired after TCZ treatment: 1 patient expired 5 months after last TCZ treatment (patient received 12 doses) from diabetic coma and 1 patient expired 9 months after last TCZ dose (patient received 6 doses) from unknown etiology. Of the 36 patients who received TCZ, no patients developed cytomegaloviremia or polyoma BK viremia (>30 copies/per).

### Sensitivity Analysis

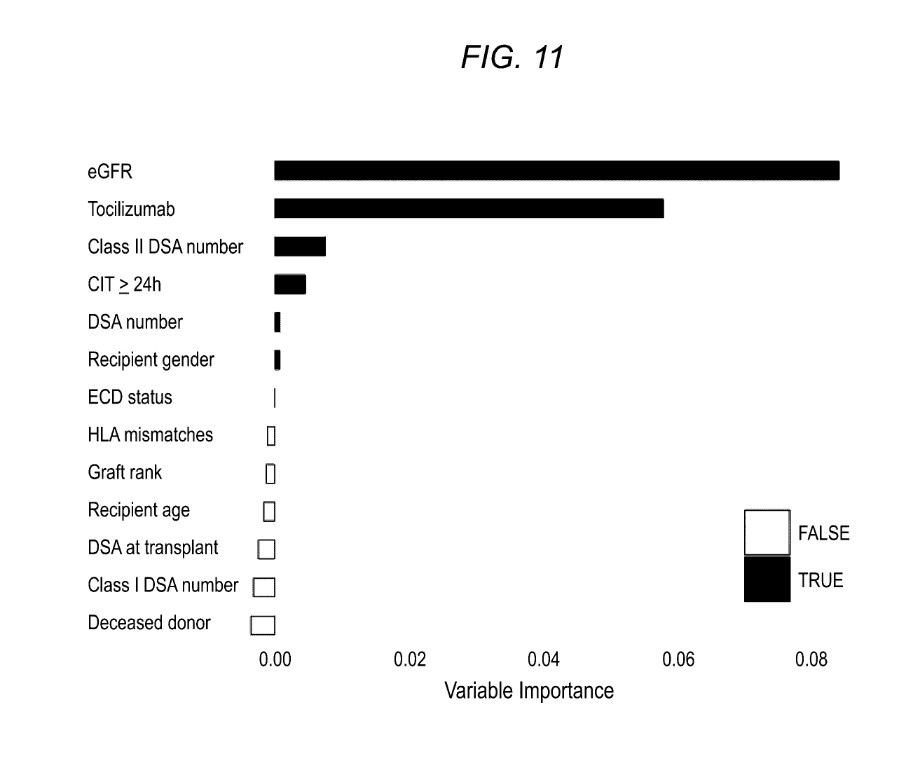

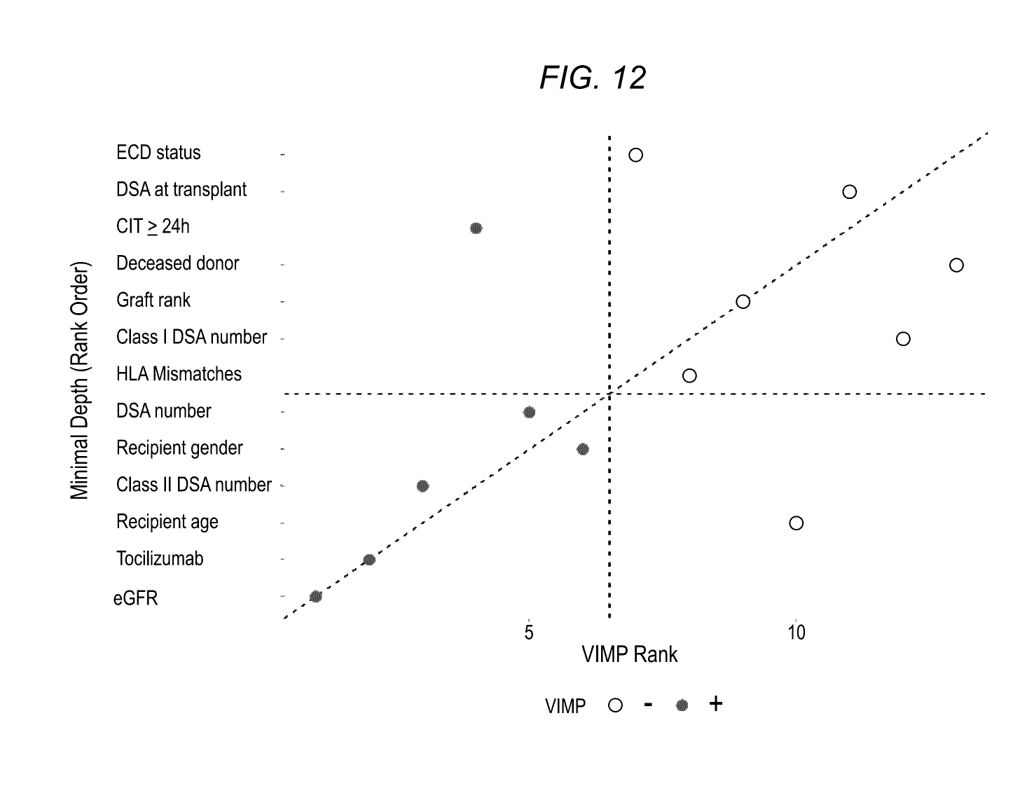

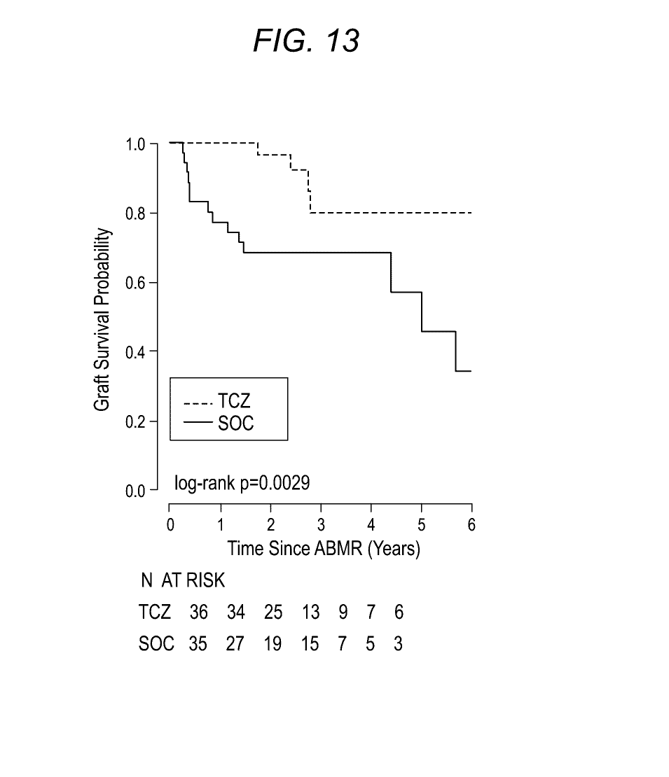

The robustness of our study was assessed using sensitivity analyses. We used VIMP measure to determine the predictors of graft loss and compared it to the minimal depth measure. The predictive variables were the eGFR, the treatment by Tocilizumab, the number of class 2 DSA, the cold ischemia time, the overall number of DSA, the recipient gender and the ECD status (FIG. 11). When the two variable selections are compared, the two methods indicate a strong relation of the treatment by Tocilizumab and eGFR to survival (FIG. 12). Because we could suppose a late action of the Tocilizumab, we studied the graft survival when graft loss was censored fort those who retained their grafts for 3 months post biopsy. There still was a significant benefit of Tocilizumab on extending graft survival. FIG. 13 still demonstrates a superior graft survival for Tocilizumab treated patients over the study period since initial biopsy. The patients treated with Tocilizumab exhibited a better graft survival (80% at 6 years post-ABMR) than the patients treated by SOC (34% at 6 years post-ABMR, log-rank test, p=0.0029).

We recently completed a trial of tocilizumab as a desensitization agent for HS patients who failed desensitization with IVIg+rituximab. This was a Phase I/II, open label study (NCT01594424). In this trial, we found that tocilizumab improved transplant rates in patients who had failed desensitization with IVIG+rituximab+/−PLEX. DSAs that were resistant to other therapies were significantly reduced with tocilizumab and none of the transplanted patients showed antibody mediated rejection in post-transplant protocol biopsies. No graft losses were seen with >24 months follow-up. Two patients subsequently developed mild ABMR 12 months after stopping tocilizumab.

Based on our experience in animal models and with desensitization of highly-HLA sensitized patients resistant to other therapies, we designed a protocol to offer Tocilizumab treatment to patients with CABMR and TG who had failed other treatment options. The outcomes for patients with CABMR and TG are poor. The results presented here with nearly 3 years of follow-up suggest Tocilzumab can alter iDSA production, stabilize allograft function, reduce inflammatory markers of ABMR and improve patient and graft survival compared to a historic group who were treated with PLEX, anti-CD20 and IVIG. Although the mechanism(s) responsible for the beneficial effects of Tocilizumab are still unclear, the outcomes are significantly better than seen with standard therapy.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A method for treating, inhibiting and/or reducing the severity of antibody mediated rejection (ABMR) of an organ transplant in a subject in need thereof comprising:
providing an inhibitor of IL-6; and
administering an effective amount of the IL-6 inhibitor to a subject in need thereof,
wherein the subject has undergone standard-of-care treatment for ABMR and the subject's response to standard-of-care treatment is ineffective.

2. A method for treating, inhibiting and/or reducing the severity of ABMR of an organ transplant in a subject in need thereof comprising:
providing an inhibitor of IL-6; and
administering an effective amount of the IL-6 inhibitor to a subject in need thereof, so as to treat ABMR of an organ transplant in the subject.

3. The method of claim 1 or 2, wherein the subject has undergone an organ transplant and exhibits symptoms of antibody mediated rejection (ABMR) of the transplanted organ.

4. The method of claim 1 or 2, wherein the IL-6 inhibitor is administered during transplant.

5. The method of claim 1 or 2, wherein the IL-6 inhibitor is administered after transplant.

6. The method of claim 2, wherein the IL-6 inhibitor is administered sequentially or simultaneously with the standard-of-care treatment.

7. The method of claim 1 or 2, wherein the organ is one or more of heart, liver, lungs, pancreas or intestines.

8. The method of claim 1 or 2, wherein the organ is the kidneys.

9. The method of claim 3, the organ is a kidney and the symptoms of ABMR are one or more of: (i) deterioration of allograft function measured by serum Creatinin and estimated Glomerular filtration rate (eGFR); (ii) presence of donor-specific antibodies; and/or (iii) biopsy evidence of capillaritis, inflammation and complement (C4d) deposition.

10. The method of claim 1 or 2, wherein the IL-6 inhibitor is selected from the group consisting of a small molecule, a peptide, an antibody or a fragment thereof and a nucleic acid molecule.

11. The method of claim 1 or 2, wherein the IL-6 inhibitor inhibits the receptor of IL-6 (IL-6R).

12. The method of claim 11, wherein the inhibitor is Tocilizumab.

13. The method of claim 12, wherein Tocilizumab is administered simultaneously or sequentially with intravenous immunoglobulin (IVIG).

14. The method of claim 1 or 6, wherein the standard-of-care treatment comprises intravenous immunoglobulin and rituximab.

15. The method of claim 1 or 2, wherein the IL-6 inhibitor is administered intravenously or subcutaneously.

16. The method of claim 1 or 2, wherein the IL-6 inhibitor is administered intravenously at a dose of about 4-8 mg/kg/month, about 3-8 mg/kg/month, about 1-4 mg/kg/month, about 1-5 mg/kg/month, about 5-10 mg/kg/month or combinations thereof.

17. The method of claim 1 or 2, wherein the IL-6 inhibitor is administered subcutaneously at a dose of about 155-170 mg every two week if the subject weighs 100 kg or less than 100 kg and about 155-170 mg per week if the subject weights more than 100 kg.

18. The method of claim 1 or 2, wherein the IL-6 inhibitor is administered for any one or more of about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, about 24 months, about 30 months, about 36 months or combinations thereof.

19. A method for reducing and/or eliminating donor specific HLA antibodies in a subject that has undergone organ transplant by the method of claim 2.

20. A method for treating, inhibiting and/or reducing the severity of ABMR post-organ transplant in highly HLA-sensitized patients by the method of claim 2.
